SEQUENCE LISTING (28 sequences)

SEQ ID NO: 1 - DNA, 1482 bp, synthetic construct
  source 1..1482: mol_type = other DNA; note = monospecific anti-PSMA CAR
atggccctgcctgtgacagccctgctgctgcctctggctctgctgctgcatgccgctagacccggatccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagccccgaggtcgaagatggattattggggccagggaaccctggtcacagtctcgagcggtggaggcggttcaggcggaggtggcagcggcggtggcgggtcaacgcagtctgtgctgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatatcgtgggcgagcaggtcgtgttcggcggagggaccaagctgaccgtcctatccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcggcgcgcctaa

SEQ ID NO: 2 - protein, 493 aa, synthetic construct
MALPVTALLLPLALLLHAARPGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKAPRSKMDYWGQGTLVTVSSGGGGSGGGGSGGGGSTQSVLTQPPSASGTPGQRVTISCSGSSSNIGSNTVNWYQQLPGTAPKLLIYSNNQRPSGVPDRFSGSKSGTSASLAISGLQSEDEADYYCAAWDIVGEQVVFGGGTKLTVLSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRA

SEQ ID NO: 3 - DNA, 1494 bp, synthetic construct
  source 1..1494: mol_type = other DNA; note = monospecific anti-Nectin-2 CAR
atgagcctgcccgtgaccgccctgctgctgcccctggccctgctgctgcacgccgccagaccaggatcccaggtgcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagatatgggtggaaggatgctatggattattggggccagggaaccctggtcacagtctcgagcggtggaggcggttcaggcggaggtggcagcggcggtggcgggtcaacgcagtctgtgttgacgcagccgccctcagtgtctgcggccccaggacagaaggtcaccatctcctgctctggaagcagctccaacattgggaataattatgtatcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatcggcccaagcagaaggtggtgttcggcggagggaccaagctgaccgtcctatccggagccaccacaaccaagcccgtgctgcggaccccaagccctgtgcaccctaccggcaccagccagcctcagagacccgaggactgccggcctcggggcagcgtgaagggcaccggcctggacttcgcctgcgacttttgggtgctggtggtggttggtggagtcctggcttgctatagcttgctagtaacagtggcctttattattttctgggtgcctgcaggaaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 4 - protein, 497 aa, synthetic construct
MSLPVTALLLPLALLLHAARPGSQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARYGWKDAMDYWGQGTLVTVSSGGGGSGGGGSGGGGSTQSVLTQPPSVSAAPGQKVTISCSGSSSNIGNNYVSWYQQLPGTAPKLLIYDNNKRPSGIPDRFSGSKSGTSATLGITGLQTGDEADYYCGTWDRPKQKVVFGGGTKLTVLSGATTTKPVLRTPSPVHPTGTSQPQRPEDCRPRGSVKGTGLDFACDFWVLVVVGGVLACYSLLVTVAFIIFWVPAGKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - DNA, 2559 bp, synthetic construct
  source 1..2559: mol_type = other DNA; note = dual PSMA Nectin-2 CAR
atggccctgcctgtgacagccctgctgctgcctctggctctgctgctgcatgccgctagacccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagccccgaggtcgaagatggattattggggccagggaaccctggtcacagtctcgagcggtggaggcggttcaggcggaggtggcagcggcggtggcgggtcaacgcagtctgtgctgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccagtctgaggatgaggctgattattactgtgcagcatgggatatcgtgggcgagcaggtcgtgttcggcggagggaccaagctgaccgtcctatccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcggcgcgccgaaggaagaggtagcctgctgacttgcggggacgtggaagagaacccagggccaatgagcctgcccgtgaccgccctgctgctgcccctggccctgctgctgcacgccgccagaccaggatcccaggtgcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagatatgggtggaaggatgctatggattattggggccagggaaccctggtcacagtctcgagcggtggaggcggttcaggcggaggtggcagcggcggtggcgggtcaacgcagtctgtgttgacgcagccgccctcagtgtctgcggccccaggacagaaggtcaccatctcctgctctggaagcagctccaacattgggaataattatgtatcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatcggcccaagcagaaggtggtgttcggcggagggaccaagctgaccgtcctatccggagccaccacaaccaagcccgtgctgcggaccccaagccctgtgcaccctaccggcaccagccagcctcagagacccgaggactgccggcctcggggcagcgtgaagggcaccggcctggacttcgcctgcgacttttgggtgctggtggtggttggtggagtcctggcttgctatagcttgctagtaacagtggcctttattattttctgggtgcctgcaggaaaacggggcagaaagaaactcctgtatatattcaaacaaccatttatgagaccagtacaaactactcaagaggaagatggctgtagctgccgatttccagaagaagaagaaggaggatgtgaactgtaa

SEQ ID NO: 6 - protein, 854 aa, synthetic construct
MALPVTALLLPLALLLHAARPGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKAPRSKMDYWGQGTLVTVSSGGGGSGGGGSGGGGSTQSVLTQPPSASGTPGQRVTISCSGSSSNIGSNTVNWYQQLPGTAPKLLIYSNNQRPSGVPDRFSGSKSGTSASLAISGLQSEDEADYYCAAWDIVGEQVVFGGGTKLTVLSGTTTPAPRPPTPAPTIASQPLSLRPEACRPAAGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAEGRGSLLTCGDVEENPGPMSLPVTALLLPLALLLHAARPGSQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARYGWKDAMDYWGQGTLVTVSSGGGGSGGGGSGGGGSTQSVLTQPPSVSAAPGQKVTISCSGSSSNIGNNYVSWYQQLPGTAPKLLIYDNNKRPSGIPDRFSGSKSGTSATLGITGLQTGDEADYYCGTWDRPKQKVVFGGGTKLTVLSGATTTKPVLRTPSPVHPTGTSQPQRPEDCRPRGSVKGTGLDFACDFWVLVVVGGVLACYSLLVTVAFIIFWVPAGKRGRKKLLYIFKQPFMRPVQTTQEEDGCSCRFPEEEEGGCEL

SEQ ID NO: 7 - DNA, 752 bp, synthetic construct
  source 1..752: mol_type = other DNA; note = anti-Nectin2 (725v5) scFv
gacaggatcccaggtgcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagatatgggtggaaggatgctatggattattggggccagggaaccctggtcacagtctcgagcggtggaggcggttcaggcggaggtggcagcggcggtggcgggtcaacgcagtctgtgttgacgcagccgccctcagtgtctgcggccccaggacagaaggtcaccatctcctgctctggaagcagctccaacattgggaataattatgtatcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatcggcccaagcagaaggtggtgttcggcggagggaccaagctgaccgtcctatccggagaca

SEQ ID NO: 8 - protein, 243 aa, synthetic construct
  source 1..243: note = anti-Nectin2 (725v5) scFv
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKAPRSKMDYWGQGTLVTVSSGGGGSGGGGSGGGGSTQSVLTQPPSASGTPGQRVTISCSGSSSNIGSNTVNWYQQLPGTAPKLLIYSNNQRPSGVPDRFSGSKSGTSASLAISGLQSEDEADYYCAAWDIVGEQVVFGGGTKLTVL

SEQ ID NO: 9 - DNA, 752 bp, synthetic construct; identical to SEQ ID NO: 7
  source 1..752: mol_type = other DNA; note = anti-Nectin2 (HRB725) scFv

SEQ ID NO: 10 - protein, 244 aa, synthetic construct
  source 1..244: note = anti-Nectin2 (HRB725) scFv
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARYGWKDAMDYWGQGTLVTVSSGGGGSGGGGSGGGGSTQSVLTQPPSVSAAPGQKVTISCSGSSSNIGNNYVSWYQQLPGTAPKLLIYDNNKRPSGIPDRFSGSKSGTSATLGITGLQTGDEADYYCGTWDRPKQKVVFGGGTKLTVL

SEQ ID NO: 11 - protein, 7 aa, synthetic construct
  source 1..7: note = CDR1-H of Nectin2_HRB725
GYTFTSY

SEQ ID NO: 12 - protein, 6 aa, synthetic construct
  source 1..6: note = CDR2-H of Nectin2_HRB725
SAYNGN

SEQ ID NO: 13 - protein, 9 aa, synthetic construct
  source 1..9: note = CDR3-H of Nectin2_HRB725
YGWKDAMDY

SEQ ID NO: 14 - protein, 13 aa, synthetic construct
  source 1..13: note = CDR1-L of Nectin2_HRB725
SGSSSNIGNNYVS

SEQ ID NO: 15 - protein, 7 aa, synthetic construct
  source 1..7: note = CDR2-L of Nectin2_HRB725
DNNKRPS

SEQ ID NO: 16 - protein, 11 aa, synthetic construct
  source 1..11: note = CDR3-L of Nectin2_HRB725
GTWDRPKQKVV

SEQ ID NO: 17 - protein, 119 aa, synthetic construct
  source 1..119: note = VH of Nectin2_HRB725
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARYGWKDAMDYWGQGTLVTVSSG

SEQ ID NO: 18 - protein, 110 aa, synthetic construct
  source 1..110: note = VL of Nectin2_HRB725
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDRPKQKVVFGGGTKLTVL

SEQ ID NO: 19 - protein, 15 aa, synthetic construct
  source 1..15: note = linker
GGGSGGGGSGGGGST

SEQ ID NO: 20 - protein, 7 aa, synthetic construct
  source 1..7: note = CDR1-H of PSMA_HRB730
GFTFSSY

SEQ ID NO: 21 - protein, 6 aa, synthetic construct
  source 1..6: note = CDR2-H of PSMA_HRB730
SGSGGS

SEQ ID NO: 22 - protein, 8 aa, synthetic construct
  source 1..8: note = CDR3-H of PSMA_HRB730
APRSKMDY

SEQ ID NO: 23 - protein, 13 aa, synthetic construct
  source 1..13: note = CDR1-L of PSMA_HRB730
SGSSSNIGSNTVN

SEQ ID NO: 24 - protein, 7 aa, synthetic construct
  source 1..7: note = CDR2-L of PSMA_HRB730
SNNQRPS

SEQ ID NO: 25 - protein, 11 aa, synthetic construct
  source 1..11: note = CDR3-L of PSMA_HRB730
AAWDIVGEQVV

SEQ ID NO: 26 - protein, 118 aa, synthetic construct
  source 1..118: note = VH of PSMA_HRB730
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKAPRSKMDYWGQGTLVTVSSG

SEQ ID NO: 27 - protein, 110 aa, synthetic construct
  source 1..110: note = VL of PSMA_HRB730
QSVLTQPPSASGTPGQRVTISCSGSSSNIGSNTVNWYQQLPGTAPKLLIYSNNQRPSGVPDRFSGSKSGTSASLAISGLQSEDEADYYCAAWDIVGEQVVFGGGTKLTVL

SEQ ID NO: 28 - protein, 20 aa, synthetic construct
  source 1..20: note = Nectin2 cancer-exposed epitope
RFTLVPSGRADGVTVTCKVE

(19) United States
(12) Patent Application Publication
(10) Pub. No.: US 2026/0356941 A1
(43) Pub. Date: Nov. 12, 2026

(54) MONOSPECIFIC AND BISPECIFIC ANTIBODIES AND ANTIBODY-DRUG CONJUGATES TARGETING NECTIN2 (CD112) AND PSMA

(71) Applicants: FONDAZIONE PER L’ISTITUTO ONCOLOGICO DI RICERCA, Bellinzona (CH); UNIVERSITE DE GENEVE, GENEVE (CH); HOPITAUX UNIVERSITAIRES DE GENEVE, GENEVE (CH)

(72) Inventors: Andrea ALIMONTI, Bosco Luganese (CH); Denis MIGLIORINI, Geneve (CH); Anna KOHL, Schwaigern (DE); Silvia BRESSAN, MONTEBELLUNA (IT)

(21) Appl. No.: 19/486,757

(22) PCT Filed: May 23, 2024
(86) PCT No.: PCT/EP2024/064291

§ 371 Date: Nov. 21, 2025

(30) Foreign Application Priority Data
May 23, 2023 (EP) 23174810.4
Jan. 30, 2024 (EP) 24154851.0

(51) Int. Cl. C07K 16/28; A61K 47/68; C07K 16/30

(52) U.S. Cl. CPC C07K 16/2803; A61K 47/6849; A61K 47/6869; A61K 47/6879; C07K 16/3069; C07K 2317/31; C07K 2317/565; C07K 2317/622; C07K 2317/77

(57) ABSTRACT

The present invention concerns anti-Nectin2 antibodies, or antigen binding fragment thereof, anti-PSMA antibodies, or antigen binding fragment thereof, bispecific antibody constructs that bind to Nectin2 and PSMA, and antibody-drug conjugates comprising such antibodies. The invention further relates to the antibody-drug conjugates for use for the treatment of cancer.

The present invention concerns anti-Nectin2 antibodies, or antigen binding fragment thereof, anti-PSMA antibodies, or antigen binding fragment thereof, bispecific antibody constructs that bind to Nectin2 and PSMA, and antibody-drug conjugates comprising such antibodies. The invention further relates to the antibody-drug conjugates for use for the treatment of cancer.

### BACKGROUND

Prostate cancer (PCa) is still a significant cause of cancer-related mortality and morbidity in men, being the second most diagnosed cancer and sixth leading cause of cancer death worldwide. Predisposition to prostate cancer development is high, with more than 70% of all cases being diagnosed in men over 65 years old. The development of prostate cancer is a multi-step process, starting with prostate hyperplasia and progressing to high-grade prostatic intraepithelial neoplasia (HGPIN) and eventually becoming invasive and metastatic.

Treatment is usually based on chemotherapy and radiotherapy with or without a castration-based strategy, but most treatments show limited duration of clinical and survival benefit due to primary and acquired resistance caused by tumor adaptability (Sumanasuriya, S. & Bono, J. D. Cold Spring Harb. Perspect. Med. 8, a030635 (2018)). Docetaxel and androgen deprivation therapy (ADT) are widely used for the treatment of prostate cancer. However, nearly all patients under ADT usually progress to castration-resistance prostate cancer (CRPC), which can eventually evolve into metastatic castration-resistant prostate cancer (mCRPC) (Nakazawa, M., Paller, C. & Kyprianou, N. Curr. Oncol. Rep. 19, 13 (2017); Saad, F. & Fizazi, K. Urology 86, 852-861 (2015)).

Therefore, current research aims at finding new therapeutic options and overcoming resistance mechanisms.

Besides, the induction of senescence, whether caused by traditional therapeutic methods or genetic mutation, plays a controversial role in cancer by both benefiting and harming tumor growth (Collado, M. Future Oncol. 6, 687-689 (2010)).

The therapeutic benefits of senescence induction come from the blockade of cellular proliferation and the activation of anti-tumoral immune responses, while the accumulation of senescent cells can stimulate tumor growth and angiogenesis, leading to relapse and therapy resistance (Hernandez-Segura, A. et al. Trends Cell Biol. 28, 436-453 (2018)). Eliminating senescent cells has become an interesting strategy for improving therapeutic efficacy. Senescent immune cells, particularly T cells, may also accumulate in the tumor microenvironment, a phenomenon known as immunosenescence, which can contribute to cancer development (Ye, J. et al. EMBO Mol. Med. 6, 1294-1311 (2014); Ye, J. et al. Blood 120, 2021-2031 (2012)). When cancer cells turn senescent, the amount of released SASP components affects the recruitment of immune cells, and the effect of SASP on immune cells populations is considered a double-edged sword effect. The recruitment of T cells, macrophages, and NK cells by SASP can prevent tumor initiation and progression, but SASP can also increase immunosuppressive myeloid cells tumor infiltration. In the absence of tumor-derived factors, myeloid cells differentiate into dendritic cells, macrophages, or neutrophils, which can contribute to immune surveillance. However, in the presence of tumor-derived factors, these myeloid cells lose their capacity to differentiate and inhibit the function of other immune cells, creating an immunotolerant environment that allows tumor progression (Ohtani, N. Inflamm. Regen. 42, 11 (2022); Eggert, T. et al. Cancer Cell 30, 533-547 (2016)). Hence, the idea of a “one-two punch” approach has been proposed, in which a stable arrest of cellular proliferation in cancer cells is established using “pro-senescence” drugs, and then subsequent treatment with an agent able to kill senescent cells (senolytic drug) is administered (Wang, C. et al. Nature 574, 268-272 (2019); Sieben C. J. et al., Trends in Cell Biology, 28, Issue 9, 723-737 (2018)). Cellular senescence represents a promising target for improving the outcome of current therapies, and several senolytic agents have been identified and obtained from natural products to be used in combination with pro-senescence drugs.

Eliminating senescent cells has therefore emerged as a promising therapeutic strategy to prevent tumor relapse and metastases in different cancers. However, senescent tumor cells are difficult to kill since they upregulate several pro-survival pathways, and the current armamentarium of effective senolytics includes only a small number of compounds that are suitable for clinical trials (Zhu, Y. et al. Aging 9, 955-963 (2017); Zhu, Y. et al. Aging Cell 15, 428-435 (2016); Yousefzadeh M J et al. EBioMedicine, 36, 18-28 (2018)).

The use of immune-checkpoint inhibitors (ICIs) has transformed cancer treatment by reactivating the immune system against malignant cells. However, only a few patients experience a durable response to ICIs, and resistance to therapy is widespread. Resistance to ICIs can be influenced by both tumor cell-extrinsic and intrinsic factors. Prostate cancer is known to have a weak immune response, with a low neoantigen load and a highly immunosuppressive microenvironment. A higher number of tumor-infiltrated lymphocytes (TILs) is a positive prognostic indicator in many types of cancer, including melanoma and breast cancer, but in prostate cancer, most of T-cells inside the tumor are CD4+ regulatory T cells (Tregs). Currently, ongoing clinical trials are evaluating the use of ICIs alone or in combination therapy for metastatic hormone-sensitive and castration-resistant prostate cancer. Immune checkpoints inhibitors (ICIs) have revolutionized the current approaches to treating cancer. In healthy condition, immune checkpoints prevent the activation of a strong immune response against normal cells in the body. This regulation is based on the specific binding of immune checkpoints expressed by immune cells to their partner binding protein. In cancer condition, the block of this interaction through ICIs results in the reactivation of the immune system against malignant cells.

Nowadays, many antibodies and small molecules targeting immune checkpoints such as CTLA-4, PD-1, PD-L1, TIGIT, TIM3 and CD47 are in clinical development. By far, the most widely used ICIs are antibodies targeting CTLA-4 (e.g. Ipilimumab), PD1 (Pembrolizumab, Nivolumab) and PD-L1 (e.g. Aterolizumab). Although the application of ICIs has improved patient outcomes across different tumor types, only a few percentages of patients achieve a durable response. Indeed, the onset of resistance mechanism to immune checkpoints inhibitors is a widespread clinical event. Even among those patients with melanoma, which present one of the highest positive response to ICIs, more than the 60% of them do not show an objective response to anti-PD-1 therapy (Ott, P. A. et al. J. Clin. Oncol. 37, 318-327 (2019)).

The cause of ICIs resistance development relies on both tumor cell-extrinsic and intrinsic factors. Among the tumor extrinsic factors, the microbiome, the PD-L1 expression levels on immune cells and the tumoral and peripheral immune cell composition play an important role in deciding the therapeutic outcome. On the other hand, epigenetic variations, mutational burden and neoantigen expression are all tumor cell-intrinsic feature which can direct the patient whether the response to ICIs therapy will be effective or not (Bagchi, S., Yuan, R. & Engleman, E. G. Annu. Rev. Pathol. 16, 223-249 (2021)).

Prostate cancer is considered as an immunologically cold disease, and a weak immune response towards it characterizes it. Indeed, it usually presents T-cell exhaustion, a low neoantigen load and a highly immunosuppressive microenvironment (de Bono, J. S. et al. Nat. Rev. Cancer 20, 455-469 (2020); Krueger, T. E., Thorek, D. L. J., Meeker, A. K., Isaacs, J. T. & Brennen, W. N. The Prostate 79, 320-330 (2019)). The tumor microenvironment is mainly constituted by Tregs, M2-polarized tumor-associated macrophages (TAMs) and myeloid-derived suppressor cells (MDSCs), and all of them generally present an immunosuppressive phenotype (Krueger, T. E., Thorek, D. L. J., Meeker, A. K., Isaacs, J. T. & Brennen, W. N. The Prostate 79, 320-330 (2019)). Specifically, MDSCs have been found to have a potent immunosuppressive role in castration-resistant prostate cancer (CRPC), where the interleukin-23 (IL-23) produced by MDSCs can regulate castration resistance by triggering androgen receptor signalling (Calcinotto, A. et al. Nature 559, 363-369 (2018)). Within this frame of reference, some pre-clinical studies already showed the beneficial effect of anti-MDSCs and ICIs combination therapy to increase the immune checkpoint blockade efficacy in CRPC models (Calcinotto, A. et al. Nature 559, 363-369 (2018); Lu, X. et al. Nature 543, 728-732 (2017)).

As previously mentioned, a higher number of tumour-infiltrated lymphocytes (TILs) is a prognostic indicator of a better outcome in many types of cancer, such as melanoma and breast cancer. In PCa, besides the low percentage of TILs, most of the T-cells inside the tumor in mCRPC resulted in being CD4+ regulatory T cells (Tregs), and the low abundant CD8+ T cells usually present an exhausted phenotype together with the expression of inhibitory receptors such as PD-1, LAG-3 and TIM-3 (Nava Rodrigues, D. et al. J. Clin. Invest. 128, 4441-4453 (2018); Brady, L. et al. Nat. Commun. 12, 1426 (2021); He, M. X. et al. Nat. Med. 27, 426-433 (2021)).

Currently, there are many ongoing clinical trials on ICIs in locally advanced or metastatic hormone-sensitive and castration-resistant prostate cancer, where ICI are used alone or in combination therapy (Rebuzzi, S. E. et al. Cancers 14, 1245 (2022)). Overall, no consistent results in terms of overall survival have been found. Patients who partially benefit from anti-PD1 therapy, such as nivolumab and pembrolizumab, were found to be characterized by a CDK12-altered tumor. Indeed, CDK12-mutated PCas are typically linked with a poor prognosis but present increased neoantigen load and lymphocytic infiltration. Besides, many studies assessing a combination therapy of ICI with standard therapies underline a better response in those patients with specific pathway aberrations (e.g. AR-V7 variant, HRD), CDK12-inactivated tumors and MSI-high tumors (Rebuzzi, S. E. et al. Cancers 14, 1245 (2022)). These clinical trials underline the need to identify novel therapeutic targets and strategies to boost the immune response in PCa patients based on the tumour's genetic signature.

Fewer than 10% of patients refractory to chemotherapy benefit from immune checkpoint blockade immunotherapy (Markowski, M. C. et al. The Prostate 80, 407-411 (2020)). Hence, mCRPC represents a critical unmet medical need.

Antibody-drug conjugates (ADC) represent an alternative strategy to ICIs. ADCs consist of a monoclonal antibody covalently linked to a cytotoxic drug through a chemical linker (Fu et al., Signal Transduct Target Ther. 2022; 7(1):9.). Through binding of the cognate tumor-associated antigen (TAA) on tumor cells, the cytotoxic drug can be delivered in a specific manner. ADCs have already shown clinical efficacy and received FDA approval for the treatment of both hematological malignancies and solid tumors (Fu et al., Signal Transduct Target Ther. 2022; 7(1):9). The major obstacle of using ADCs in solid tumors is the poor availability of tumour-specific targets. Most solid tumor TAAs are also expressed on non-malignant primary cells in critical tissues, even at a lower level. The main side effects comprise hematotoxicity including neutropenia, thrombocytopenia, leukopenia and anemia (Fu et al., Signal Transduct Target Ther. 2022; 7(1):9). For example, through changes in the Fc domain of the mAb, these side effects can be alleviated (Oshima et al., 2018).

Currently, several preclinical studies (Boinapally et al., Eur J Nucl Med Mol Imaging, 2022, 49(13):4369-4381; Machulkin et al., Eur J Med Chem. 2022 Jan. 5; 227:113936) as well as clinical trials (Cho et al., Mol Cancer Ther. 2018; 17(10):2176-2186), Milowsky et al., Urol Oncol., 2016; 34(12):530.e15-530.e21; Petrylak et al., Prostate. 2020 January; 80(1):99-108) are ongoing employing PSMA-targeting ADCs for the treatment of mCRPC. In preclinical studies, the respective ADCs showed strong and specific cytotoxicity. Unfortunately, in clinical trials, they show only limited anti-tumor activity, accompanied by common treatment-related adverse effects such as neutropenia and neuropathy. Thus, the utility of this therapeutic strategy may be limited by multiple factors. First, heterogeneous expression and/or downregulation of PSMA can cause tumor escape, challenging pharmacokinetics of the adjacent drug, and the commonly observed side effects.

Understanding the adaptive immune response in advanced prostate cancer (APC) has become crucial, especially after the anti-CTLA4 (ipilimumab) therapy failed in phase Ill trials (Beer, T. M. et al. J. Clin. Oncol. Off. J. Am. Soc. Clin. Oncol. 35, 40-47 (2017)). Also, PD-L1, the main target of existing immunotherapies, is infrequently expressed on prostate cancer (Haffner, M. C. et al. Am. J. Pathol. 188, 1478-1485 (2018)). Lately, the largest clinical trial testing anti-CTLA-4 plus anti-PD-1 in CRPC did not significantly ameliorate the patient's clinical outcome (Sharma, P. et al. Cancer Cell 38, 489-499.e3 (2020)).

### SUMMARY OF THE INVENTION

The inventors hypothesized that further development of effective ADCs for CRPC should include the identification of robust tumor target antigens that are stably and homogeneously expressed, incorporating safety mechanisms to mitigate toxicity, as well as strategies to overcome premature release of cytotoxic payloads in the circulation.

The inventors therefore aimed to identify novel immune checkpoints in prostate cancer and exploit them as a new target for developing immunotherapeutic strategies for prostate cancer. Through analysis of bulk-RNA and single-cell data from human patients, the transmembrane protein Nectin2 was identified as upregulated in epithelial prostate cancer cells. Nectin2 has immunomodulatory functions and interacts with receptors expressed by T and NK cells. The upregulation of Nectin2 was found to be even higher upon senescence induction in vitro and in vivo models of therapy-induced senescence and PTEN-loss—induced cellular senescence.

Based on these findings, Nectin2 represents an exploitable target to remove senescent cancer cells from the tumor and reactivate the immune response. Novel scFvs were isolated using a human phage-display library against the extracellular domains of Nectin2 and PSMA, and candidates were selected upon binding to Nectin2 and/or PSMA expressing human PCa cell lines. Their specificity was further confirmed using T cells modified to harbor a chimeric antigen receptor (CAR) incorporating the selected anti-Nectin2 or anti-PSMA scFvs. Furthermore, we observed selective binding of the novel anti-Nectin2 scFv to a cancer-exposed epitope of Nectin2, sparing non-malignant Nectin2-expressing human cells (in vitro). Importantly, the anti-Nectin2 scFv-Fc is internalized into human PCa cells upon staining. Thus, using these validated scFvs, senolytic bispecific ADCs targeting Nectin2 and PSMA will be developed.

The invention thus relates to anti-Nectin2 antibody, or antigen binding fragment thereof, that comprises (i) a variable heavy chain domain (V_{H}) comprising a CDR1-H of sequence SEQ ID NO: 11, a CDR2-H of sequence SEQ ID NO: 12, and a CDR3-H of sequence SEQ ID NO: 13, and (ii) a variable light chain domain (V_{L}) comprising a CDR1-L of sequence SEQ ID NO: 14, a CDR2-L of sequence SEQ ID NO: 15, and a CDR3-L of sequence SEQ ID NO: 16.

In some embodiments the anti-Nectin2 antibody, or antigen binding fragment thereof, is a scFV.

The invention further relates to a polypeptide comprising the anti-Nectin2 scFv of the invention.

Also provided is a pair of isolated nucleic acids comprising a sequence encoding respectively the variable heavy chain domain (V_{H}) and variable light chain domain (V_{L}) of the anti-Nectin2 antibody, or antigen binding fragment thereof.

Also provided is an isolated nucleic acid comprising a sequence encoding the anti-Nectin2 scFv of the invention, or the polypeptide comprising said anti-Nectin2 scFv.

The invention further relates to an antibody-drug conjugate (ADC) comprising (i) the anti-Nectin2 antibody, or antigen binding fragment thereof, according to the invention, (ii) a cytotoxic payload, and (iii) a linker connecting the anti-Nectin2 antibody, or antigen binding fragment thereof, and the cytotoxic payload.

The invention also relates to an anti-PSMA antibody, or antigen binding fragment thereof, that comprises (i) a variable heavy chain domain (V_{H}) comprising a CDR1-H of sequence SEQ ID NO: 20, a CDR2-H of sequence SEQ ID NO: 21, and a CDR3-H of sequence SEQ ID NO: 22, and (ii) a variable light chain domain (V_{L}) comprising a CDR1-L of sequence SEQ ID NO: 23, a CDR2-L of sequence SEQ ID NO: 24, and a CDR3-L of sequence SEQ ID NO: 25.

In some embodiments the anti-PSMA antibody, or antigen binding fragment thereof, is a scFV.

The invention further relates to a polypeptide comprising the anti-PSMA scFv of the invention.

Also provided is a pair of isolated nucleic acids comprising a sequence encoding respectively the variable heavy chain domain (V_{H}) and variable light chain domain (V_{L}) of the anti-PSMA antibody, or antigen binding fragment thereof.

Also provided is an isolated nucleic acid comprising a sequence encoding the anti-PSMA scFv of the invention, or the polypeptide comprising said anti-PSMA scFv.

The invention further relates to an antibody-drug conjugate (ADC) comprising (i) the anti-PSMA antibody, or antigen binding fragment thereof, according to the invention, (ii) a cytotoxic payload, and (iii) a linker connecting the anti-Nectin2 antibody, or antigen binding fragment thereof, and the cytotoxic payload.

Further provided is a bispecific antibody construct that binds at least to Nectin2 and PSMA, and an ADC comprising said bispecific antibody, a cytotoxic payload, and a linker connecting the bispecific antibody construct and the cytotoxic payload.

The invention further relates to an ADC according to the invention for use for the treatment of cancer, and to a pharmaceutical composition comprising such an ADC.

### DETAILED DESCRIPTION

### Definitions

“Prostate-specific membrane antigen” or “PSMA” is a type II membrane protein expressed in all forms of prostate tissue. A reference sequence of human PSMA is available in Uniprot database, under accession number Q04609 (entry version 222 of 3 May 2023).

“Nectin2” denotes a type I membrane glycoprotein with two Ig-like C2-type domains and an Ig-like V-type domain, which is a plasma membrane component of adherens junctions. A reference sequence of human Nectin2 is available in Uniprot database, under accession number Q92692 (entry version 217 of 3 May 2023). Nectin2 is also named Poliovirus Receptor-Related Protein-2, Poliovirus Receptor-Like 2, CD112, or PRR-2.

An “antibody” may be a natural or conventional immunoglobulin molecule in which disulfide bonds link two heavy chains to each other and each heavy chain is linked to a light chain by a disulfide bond. The light chain includes two domains or regions, a variable domain (VL) and a constant domain (CL). The heavy chain includes four domains, a variable domain (VH) and three constant domains (CH1, CH2 and CH3, collectively referred to as CH). The specificity of the antibody resides in the structural complementarity between the antibody combining site and the antigenic determinant. Antibody combining sites are made up of residues that are primarily from the hypervariable or “complementarity determining regions” (CDRs). The light and heavy chains of an immunoglobulin each have three CDRs, designated CDR-L1, CDR-L2, CDR-L3 and CDR-H1, CDR-H2, CDR-H3, respectively. A conventional antibody antigen-binding site, therefore, includes six CDRs, comprising the CDR set from each of a heavy and a light chain V region.

“Framework Regions” (FRs) refer to amino acid sequences interposed between CDRs, i.e. to those portions of immunoglobulin light and heavy chain variable regions that are relatively conserved among different immunoglobulins in a single species. The light and heavy chains of an immunoglobulin each have four FRs, designated FR-L1, FR-L2, FR-L3, FR-L4, and FR-H1, FR-H2, FR-H3, FR-H4, respectively.

In the context of the invention, CDR/FR definition in an immunoglobulin light or heavy chain is to be determined based on Chothia numbering (Chothia et al., J Mol Biol. 1987; 196(4):901-17).

As used herein, the term “antibody” denotes conventional antibodies and antigen binding fragments thereof, as well as chimeric, humanised, bispecific or multispecific antibodies.

The term “monoclonal antibody” or “mAb” as used herein refers to an antibody molecule of a single primary structure that is directed against a specific antigen, and is not to be construed as requiring production of the antibody by any particular method. A monoclonal antibody may be produced by a single clone of B cells or hybridoma, but may also be recombinant, i.e. produced by protein engineering.

“Fragments” of (conventional) antibodies comprise a portion of an intact antibody, in particular the antigen binding region or variable region of the intact antibody. Examples of antibody fragments include Fv, Fab, F(ab′)2, Fab′, dsFv, (dsFv)2, scFv, sc(Fv)2, diabodies, bispecific and multispecific antibodies formed from antibody fragments.

The antibody or antibody fragment can be an isotype or subtype; preferably is an IgG, still preferably an IgG1.

The term “bispecific antibody” refers to an antibody having the capacity to bind to two distinct epitopes either on a single antigen or two different antigens. A bispecific antibody of the present invention may be bivalent, trivalent, or tetravalent. As used herein, “valent”, “valence”, “valencies”, or other grammatical variations thereof, mean the number of antigen binding sites in an antibody molecule. These antigen recognition sites may recognize the same epitope or different epitopes.

As used herein, the percentage of identity is calculated after pairwise global sequence alignment with Smith-Waterman algorithm, for instance using EMBOSS Needle with default settings (matrix BLOSUM62, gap open 10, gap extend 0.5, end gap penalty false, end gap open 10, end gap extend 0.5).

Throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”). Furthermore the indefinite article “a” or “an” does not exclude a plurality.

### Anti-Nectin2 Antibodies

The invention relates to an anti-Nectin2 antibody, or antigen binding fragment thereof, that comprises (i) a variable heavy chain domain (V_{H}) comprising a CDR1-H of sequence GYTFTSY (SEQ ID NO: 11), a CDR2-H of sequence SAYNGN (SEQ ID NO: 12), and a CDR3-H of sequence YGWKDAMDY (SEQ ID NO: 13), and (ii) a variable light chain domain (V_{L}) comprising a CDR1-L of sequence SGSSSNIGNNYVS (SEQ ID NO: 14), a CDR2-L of sequence DNNKRPS (SEQ ID NO: 15), and a CDR3-L of sequence GTWDRPKQKVV (SEQ ID NO: 16).

The anti-Nectin2 antibody or antibody fragment may be a recombinant antibody, a monoclonal antibody, a chimeric antibody, a humanized antibody, a human antibody.

Is some embodiments, the anti-Nectin2 antibody, or antigen binding fragment thereof, comprises:
(a) a V_{H} that comprises sequence SEQ ID NO: 17 or a sequence at least 80%, 85%, 90%, 95%, or 98% identical thereto,
(b) a V_{L} that comprises sequence SEQ ID NO: 18 or a sequence at least 80%, 85%, 90%, 95%, or 98% identical thereto, or
(c) a V_{H} that comprises sequence SEQ ID NO: 17 or a sequence at least 80%, 85%, 90%, 95%, or 98% identical thereto, and a V_{L} that comprises sequence SEQ ID NO: 18 or a sequence at least 80%, 85%, 90%, 95%, or 98% identical thereto.

In some embodiments, the anti-Nectin2 antibody, or antigen binding fragment thereof, binds to a cancer-exposed epitope of Nectin2. The anti-Nectin2 antibody, or antigen binding fragment thereof, therefore discriminates cancerous Nectin2-expressing human cells and non-malignant Nectin2-expressing human cells. In some embodiments, the anti-Nectin2 antibody, or antigen binding fragment thereof, binds to an epitope comprising or consisting of sequence SEQ ID NO: 28.

In some embodiments, the anti-Nectin2 antibody, or antigen binding fragment thereof is a single chain variable fragment (scFv).

In some embodiments, the anti-Nectin2 scFv comprises (i) a V_{H} comprising a CDR1-H of sequence SEQ ID NO: 11, a CDR2-H of sequence SEQ ID NO: 12, and a CDR3-H of sequence SEQ ID NO: 13, and (ii) a V_{L} comprising a CDR1-L of sequence SEQ ID NO: 14, a CDR2-L of sequence SEQ ID NO: 15, and a CDR3-L of sequence SEQ ID NO: 16, and (iii) a linker connecting the V_{H} and V_{L}

In some embodiments, the V_{H} of the anti-Nectin2 scFv comprises sequence SEQ ID NO: 17 or a sequence at least 80%, 85%, 90%, 95%, or 98% identical thereto.

In some embodiments, the V_{L} of the anti-Nectin2 scFv comprises sequence SEQ ID NO: 18 or a sequence at least 80%, 85%, 90%, 95%, or 98% identical thereto.

In some embodiments, the V_{H} of the anti-Nectin2 scFv comprises sequence SEQ ID NO: 17 or a sequence at least 80%, 85%, 90%, 95%, or 98% identical thereto, and the V_{L} of the anti-Nectin2 scFv comprises sequence SEQ ID NO: 18 or a sequence at least 80%, 85%, 90%, 95%, or 98% identical thereto.

The linker is a chemical linker or a polypeptide linker. In some embodiments, the linker connecting the VH and VL of the anti-Nectin2 scFv comprises sequence GGGSGGGGSGGGGST (SEQ ID NO: 19), or a sequence at least 80%, 85%, 90%, 95%, or 98% identical thereto.

Preferably, the anti-Nectin2 scFv comprises or consists of sequence SEQ ID NO: 10.

A polypeptide comprising the anti-Nectin2 scFv also makes art of the invention.

In some embodiments, said polypeptide is not a chimeric antigen receptor (CAR), in particular not CAR that comprises an antigen binding domain that binds Nectin2, or that comprises an antigen binding domain that binds PSMA and an antigen binding domain that binds Nectin2.

The invention further relates to a pair of nucleic acids encoding respectively the V_{H} and V_{L} chain of an anti-Nectin2 antibody, or antibody fragment, as disclosed herein. The invention further relates to a nucleic acid comprising a sequence encoding the anti-Nectin2 scFv, or the polypeptide comprising the anti-Nectin2 scFv.

In some embodiments, the nucleic acid comprises sequence SEQ ID NO: 9, or a sequence at least 80%, 85%, 90%, 95%, or 98% identical thereto.

### Anti-PSMA Antibodies

The invention relates to an anti-PSMA antibody, or antigen binding fragment thereof, that comprises (i) a variable heavy chain domain (V_{H}) comprising a CDR1-H of sequence GFTFSSY SEQ ID NO: 20, a CDR2-H of sequence SGSGGS SEQ ID NO: 21, and a CDR3-H of sequence APRSKMDY SEQ ID NO: 22, and (ii) a variable light chain domain (V_{L}) comprising a CDR1-L of sequence SGSSSNIGSNTVN SEQ ID NO: 23, a CDR2-L of sequence SNNQRPS SEQ ID NO: 24, and a CDR3-L of sequence AAWDIVGEQVV SEQ ID NO: 25.

The anti-PSMA antibody or antibody fragment may be a recombinant antibody, a monoclonal antibody, a chimeric antibody, a humanized antibody, a human antibody.

In some embodiments, the anti-PSMA antibody, or antigen binding fragment thereof, comprises:
(a) a V_{H} that comprises sequence SEQ ID NO: 26 or a sequence at least 80%, 85%, 90%, 95%, or 98% identical thereto,
(b) a V_{L} that comprises sequence SEQ ID NO: 27 or a sequence at least 80%, 85%, 90%, 95%, or 98% identical thereto, or
(c) a V_{H} that comprises sequence SEQ ID NO: 26 or a sequence at least 80%, 85%, 90%, 95%, or 98% identical thereto, and a V_{L} that comprises sequence SEQ ID NO: 27 or a sequence at least 80%, 85%, 90%, 95%, or 98% identical thereto.

In some embodiments, the anti-PSMA antibody, or antigen binding fragment thereof is a single chain variable fragment (scFv).

In some embodiments, the anti-PSMA scFv comprises (i) a V_{H} comprising a CDR1-H of sequence SEQ ID NO: 20, a CDR2-H of sequence SEQ ID NO: 21, and a CDR3-H of sequence SEQ ID NO: 22, and (ii) a V_{L} comprising a CDR1-L of sequence SEQ ID NO: 23, a CDR2-L of sequence SEQ ID NO: 24, and a CDR3-L of sequence SEQ ID NO: 25, and (iii) a linker connecting the V_{H} and V_{L}In some embodiments, the V_{H} of the anti-PSMA scFv comprises sequence SEQ ID NO: 26 or a sequence at least 80%, 85%, 90%, 95%, or 98% identical thereto.

In some embodiments, the V_{L} of the anti-PSMA scFv comprises sequence SEQ ID NO: 27 or a sequence at least 80%, 85%, 90%, 95%, or 98% identical thereto.

In some embodiments, the V_{H} of the anti-PSMA scFv comprises sequence SEQ ID NO: 26 or a sequence at least 80%, 85%, 90%, 95%, or 98% identical thereto, and the V_{L} of the anti-PSMA scFv comprises sequence SEQ ID NO: 27 or a sequence at least 80%, 85%, 90%, 95%, or 98% identical thereto.

The linker is a chemical linker or a polypeptide linker. In some embodiments, the linker connecting the VH and VL of the anti-PSMA scFv comprises sequence GGGSGGGGSGGGGST (SEQ ID NO: 19), or a sequence at least 80%, 85%, 90%, 95%, or 98% identical thereto.

Preferably, the anti-PSMA scFv comprises or consists of sequence SEQ ID NO: 8.

A polypeptide comprising the anti-PSMA scFv also makes art of the invention.

In some embodiments, said polypeptide is not a CAR, in particular not CAR that comprises an antigen binding domain that binds PSMA, or that comprises an antigen binding domain that binds PSMA and an antigen binding domain that binds Nectin2.

The invention further relates to a pair of nucleic acids encoding respectively the V_{H} and V_{L} chain of an anti-PSMA antibody, or antibody fragment, as disclosed herein. The invention further relates to a nucleic acid comprising a sequence encoding the anti-PSMA scFv, or the polypeptide comprising the anti-PSMA scFv.

In some embodiments, the nucleic acid comprises sequence SEQ ID NO: 7, or a sequence at least 80%, 85%, 90%, 95%, or 98% identical thereto.

### Multispecific Antibodies Binding to Nectin2 and PSMA

Also provided are multispecific (e.g. bispecific) antibody constructs that bind at least to Nectin2 and PSMA. Such multispecific or bispecific antibody constructs typically comprise (a) a first antigen binding moiety that specifically binds Nectin2, and (b) a second antigen binding moiety that specifically binds PSMA, wherein the first and second antigen binding moieties are connected directly or by a linker.

In some embodiments, the multispecific or bispecific antibody construct comprises an anti-Nectin2 antibody, or antigen binding fragment thereof, and an anti-PSMA antibody, or antigen binding fragment thereof, as herein disclosed. In some embodiments, the anti-Nectin2 antibody, or antigen binding fragment thereof, and the anti-PSMA antibody, or antigen binding fragment thereof, are connected by a linker.

In some embodiments, in the multispecific or bispecific antibody construct:
[A] the first antigen binding moiety that specifically binds Nectin2 comprises (i) a variable heavy chain domain (V_{H}) comprising a CDR1-H of sequence SEQ ID NO: 11, a CDR2-H of sequence SEQ ID NO: 12, and a CDR3-H of sequence SEQ ID NO: 13, and (ii) a variable light chain domain (V_{L}) comprising a CDR1-L of sequence SEQ ID NO: 14, a CDR2-L of sequence SEQ ID NO: 15, and a CDR3-L of sequence SEQ ID NO: 16; or
[B] the second antigen binding moiety that specifically binds PSMA comprises (i) a variable heavy chain domain (V_{H}) comprising a CDR1-H of sequence SEQ ID NO: 20, a CDR2-H of sequence SEQ ID NO: 21, and a CDR3-H of sequence SEQ ID NO: 22, and (ii) a variable light chain domain (V_{L}) comprising a CDR1-L of sequence SEQ ID NO: 23, a CDR2-L of sequence SEQ ID NO: 24, and a CDR3-L of sequence SEQ ID NO: 25; or
[C] the first antigen binding moiety that specifically binds Nectin2 comprises (i) a variable heavy chain domain (V_{H}) comprising a CDR1-H of sequence SEQ ID NO: 11, a CDR2-H of sequence SEQ ID NO: 12, and a CDR3-H of sequence SEQ ID NO: 13, and (ii) a variable light chain domain (V_{L}) comprising a CDR1-L of sequence SEQ ID NO: 14, a CDR2-L of sequence SEQ ID NO: 15, and a CDR3-L of sequence SEQ ID NO: 16; and
the second antigen binding moiety that specifically binds PSMA comprises (i) a variable heavy chain domain (V_{H}) comprising a CDR1-H of sequence SEQ ID NO: 20, a CDR2-H of sequence SEQ ID NO: 21, and a CDR3-H of sequence SEQ ID NO: 22, and (ii) a variable light chain domain (V_{L}) comprising a CDR1-L of sequence SEQ ID NO: 23, a CDR2-L of sequence SEQ ID NO: 24, and a CDR3-L of sequence SEQ ID NO: 25; or
[D] the first antigen binding moiety that specifically binds Nectin2 comprises (i) a variable heavy chain domain (V_{H}) comprising a CDR1-H of sequence SEQ ID NO: 11, a CDR2-H of sequence SEQ ID NO: 12, and a CDR3-H of sequence SEQ ID NO: 13, and (ii) a variable light chain domain (V_{L}) comprising a CDR1-L of sequence SEQ ID NO: 14, a CDR2-L of sequence SEQ ID NO: 15, and a CDR3-L of sequence SEQ ID NO: 16; and
the second antigen binding moiety that specifically binds PSMA comprises a V_{H} that comprises sequence SEQ ID NO: 26 or a sequence at least 80% identical thereto, and/or a V_{L} that comprises sequence SEQ ID NO: 27 or a sequence at least 80% identical thereto;
[E] the first antigen binding moiety that specifically binds Nectin2 comprises a V_{H} that comprises sequence SEQ ID NO: 17 or a sequence at least 80% identical thereto, and/or a V_{L} that comprises sequence SEQ ID NO: 18 or a sequence at least 80% identical thereto; and
the second antigen binding moiety that specifically binds PSMA comprises (i) a variable heavy chain domain (V_{H}) comprising a CDR1-H of sequence SEQ ID NO: 20, a CDR2-H of sequence SEQ ID NO: 21, and a CDR3-H of sequence SEQ ID NO: 22, and (ii) a variable light chain domain (V_{L}) comprising a CDR1-L of sequence SEQ ID NO: 23, a CDR2-L of sequence SEQ ID NO: 24, and a CDR3-L of sequence SEQ ID NO: 25; or
[F] the first antigen binding moiety that specifically binds Nectin2 comprises a V_{H} that comprises sequence SEQ ID NO: 17 or a sequence at least 80% identical thereto, and/or a V_{L} that comprises sequence SEQ ID NO: 18 or a sequence at least 80% identical thereto; and
the second antigen binding moiety that specifically binds PSMA comprises a V_{H} that comprises sequence SEQ ID NO: 26 or a sequence at least 80% identical thereto, and/or a V_{L} that comprises sequence SEQ ID NO: 27 or a sequence at least 80% identical thereto; or
[G] the first antigen binding moiety that specifically binds Nectin2 comprises a V_{H} that comprises sequence SEQ ID NO: 17 or a sequence at least 80% identical thereto, and a V_{L} that comprises sequence SEQ ID NO: 18 or a sequence at least 80% identical thereto; and
the second antigen binding moiety that specifically binds PSMA comprises a V_{H} that comprises sequence SEQ ID NO: 26 or a sequence at least 80% identical thereto, and a V_{L} that comprises sequence SEQ ID NO: 27 or a sequence at least 80% identical thereto.

In the multispecific or bispecific antibody constructs, the linker is a chemical linker or a polypeptide linker.

In some embodiments, the multispecific or bispecific antibody construct comprises an anti-Nectin2 antibody and an anti-PSMA antibody, as herein disclosed. IgG-Based bispecific antibodies are similar in structure to native antibodies, and all have Fc regions.

Different IgG-Based bispecific antibody formats are available and include knobs-into-holes format, or CrossMab format.

In some embodiments, the multispecific or bispecific antibody construct comprises an anti-Nectin2 antibody fragment and an anti-PSMA antibody fragment, as herein disclosed.

In some embodiments, the multispecific or bispecific antibody construct comprises an anti-Nectin2 scFv and an anti-PSMA scFv, as herein disclosed.

Fragment-based bispecific antibodies are composed of the variable light and heavy domains from two antibodies, or the Fab units, and lack the Fc region. These fragments are bound together by linkers (e.g., disulfide bonds or non-covalent interactions). Different Fragment-based bispecific antibodies are available and include bispecific T-cell engager (BiTE), TCR-mimic, tandem diabody (TandAb), dual affinity retargeting (DART), or bi-nanobody (Ma et al., Front Immunol. 2021; 12: 626616).

### Antibody Drug Conjugates

The invention further relates to antibody-drug conjugates that comprise an antibody or antibody fragment as defined herein.

An antibody-drug conjugate (ADC) typically comprises (i) an antibody, or antigen binding fragment thereof, (ii) a cytotoxic payload, and (iii) a linker connecting the antibody, or antigen binding fragment thereof, and the cytotoxic payload.

In some embodiments, the ADC comprises (i) an anti-Nectin2 antibody, or antigen binding fragment thereof, as disclosed herein, (ii) a cytotoxic payload, and (iii) a linker connecting the anti-Nectin2 antibody, or antigen binding fragment thereof, and the cytotoxic payload.

In some embodiments, the ADC comprises (i) an anti-PSMA antibody, or antigen binding fragment thereof, as disclosed herein, (ii) a cytotoxic payload, and (iii) a linker connecting the anti-PSMA antibody, or antigen binding fragment thereof, and the cytotoxic payload.

In some embodiments, the ADC comprises (i) a multispecific or bispecific antibody construct that binds at least to Nectin2 and PSMA, (ii) a cytotoxic payload, and (iii) a linker connecting the bispecific antibody construct and the cytotoxic payload. In some embodiments, the multispecific or bispecific antibody construct that binds at least to Nectin2 and PSMA comprises an anti-Nectin2 antibody, or antigen binding fragment thereof, as herein disclosed, and an anti-PSMA antibody, or antigen binding fragment thereof, as herein disclosed.

Suitable ADC linkers include cleavable and non-cleavable linkers.

In some embodiments, the linker is a cleavable linker. Such likers typically include chemical cleavage linkers (e.g. hydrazone bond and disulfide bond) and enzyme cleavage linkers (e.g. glucuronide bond and peptide bond).

In some embodiments, the linker is a non-cleavable linker (e.g., a thioether or maleimidocaproyl group).

Non limiting examples of cytotoxic payloads that are conventionally incorporated in ADCs include tubulin inhibitors (e.g. auristatin derivatives such as monomethyl auristatin E (MMAE) and monomethyl auristatin F (MMAF)), DNA damaging agents (e.g. inducing DNA double strand break, such as calicheamicins; DNA alkylation, such as duocarmycins; DNA intercalation, such as topoisomerase I inhibitors; DNA crosslink, such as pyrrolobenzodiazepines (PBD)), and immunomodulators (such as TLR agonists). Cytotoxic payloads also include radioligands such as Actinium-225, Lead-212, Gallium-68, Lutetium-177, Radium-223, Zirconium-89.

### Therapeutic Uses and Pharmaceutical Compositions

The ADCs according to the invention are for use as a medicament, in particular for use for treating prostate cancer, in particular is a castration-resistant prostate cancer, or metastatic castration-resistant prostate cancer.

The invention further relates to a method of treatment of cancer in a subject in need thereof, which comprises administering to the subject an ADC as herein disclosed.

A pharmaceutical composition comprising an ADC as herein disclosed, and a pharmaceutically acceptable carrier.

The subject may be a mammal, such as a primate (e.g. human, monkey), a rodent (e.g. rat, mouse), a canine (e.g. a dog), or a feline (e.g. a cat). Preferably, the subject is a human.

In some embodiments at least one prostate cancer therapeutic agent is used in combination with the ADC. Said prostate cancer therapeutic agent may be selected from the group consisting of chemotherapeutic agents (such as Docetaxel, or Enzalutamide), and immune checkpoint inhibitors (such as anti-PD1 or anti-TIGIT antibodies).

The invention will be further illustrated in view of the following figures and examples.

### FIGURES

FIG. 1. Nectin2-PVRIG axis is upregulated in PCa. (A) mRNA expression level of different immune checkpoint expressed by epithelial and immune cells from bulk-RNA data. (B) Schematic representation of the Nectin2 and PVR interaction with receptor expressed by T and NK cells. (C) Gene expression level on human prostate cancer samples. Among Nectin-like molecules, Nectin2 is the most abundant one in epithelial cells. On the other hand, PVRIG is the most expressed receptor in immune cells.

FIG. 2. Nectin2 is upregulated in Pten-loss-induced cellular senescence (PICS). (A) Experimental design and Volcano plot of proteins differentially present between FDG+ and FDG− epithelial cells. (B) Results of western blot analysis in wild type and pten−/− prostate samples and quantification of the fold change compared to wild type of Nectin2 and p21. (C) Real time PCR data of Nectin2 and p16 mRNA expression level in FDG+ and FDG- sorted epithelial prostate tumor cells. (D) Nectin2 expression by RNA-seq data on wild type, pten−/− and pten−/−p53−/− mice. (E) PTEN null mice were treated with Docetaxel 10 mg/Kg once a week for four weeks. After 4 weeks, the prostate was collected, and senescence induction was quantified through beta-galactosidase assay on OCT embedded tissue. (F) Anterior lobe size was significantly reduced in treated mice compared to untreated one and (G) real time PCR on prostate samples confirmed Nectin2 upregulation.

FIG. 3. Therapy-induced senescence (TIS) further enhances the level of Nectin2 in PCa. (A) Senescence induction in PC3, 22RV1 and LNCaP human prostate cancer cell lines using Palbociclib and Docetaxel. Treatments were added in the medium for three days, then the medium was changed and after three days cells were seeded for beta-galactosidase assay. (B) Nectin2 expression in human prostate cancer cell lines after senescence induction using Palbociclib or Docetaxel by real-time and (C) flow cytometry. (D) Senescence induction upon therapy induced senescence measured through FDG staining in 22rv1 cells and (E) mean fluorescence intensity (MFI) of Nectin2 in untreated and treated cells. (F) MFI of Nectin2 cells in FDG- and FDG+ population and percentage of FDG- and FDG+ LNCAP cells untreated or cultured in androgen deprivation (ADT). (G) Results of beta-galactosidase assay in LNCAP upon senescence induction using androgen deprivation and (H) Nectin2 mRNA level.

FIG. 4. Nectin2 and PSMA expression correlates in PCa. (A) Correlation matrix showing Pearson coefficients in human RNA seq samples. (B) Scatter plot representing correlation between Nectin2 and FOLH1, and PVR and FOLH1. (C) Percentage of Nectin2+PSMA−, Nectin2+PSMA+, Nectin2-PSMA+ and Nectin2-PSMA- in tumor cells. (D) Comparison of the percentage of Nectin2+PSMA−, Nectin2+PSMA+, Nectin2-PSMA+ and Nectin2-PSMA− in tumor cells in castration-sensitive and castration-resistance prostate cancer patients. (E) Western blot on patient-derived organoid shows a correlation between Nectin2 and PSMA. (F) PSMA protein expression level by western blot on human prostate cancer cell lines upon TIS.

FIG. 5. Anti-PSMA and anti-Nectin2 combinatorial CAR T cells specifically lyse PSMA+/Nectin2+ and PSMA+/Nectin2− human PCa cell lines. (A) Target antigen expression on human PCa cell lines as assessed via flow cytometry. (B) CAR design for second generation monospecific anti-PSMA and anti-Nectin2 CAR constructs. (C) CAR design for combinatorial CARs targeting PSMA and Nectin2. (D) CAR expression on T cells versus non-transduced (NTD) as assessed by flow cytometry. (E-F) CombiCAR T cells specifically lyse PSMA+/Nectin2+ and PSMA+/Nectin2− human PCa cells in 92-hr fluorescence-based lysis assay. (G) ELISA determination of the secretion of IFNg by CAR T cells in co-culture with tumor cells as described in (E-F).

FIG. 6. Identification of novel anti-Nectin2 and anti-PSMA scFvs with high tumor-specificity and internalization capacity. (A) Quantification of immunofluorescence staining from human non-malignant cells (HEK293T, HUVEC, HK2, BPH, RWPE, MCF10A) and human prostate cancer cell lines (22rv1n LNCAP, PC3, PC3 Nectin2 KO, DU145), using a commercial anti-Nectin2 antibody, or the anti-Nectin2 scFv antibody, respectively. (B) Immunofluorescence staining of LNCaP cells using a secondary anti-human Fc antibody and quantification of detected fluorescence intensity. Cells were stained with the anti-Nectin2 scFv-Fc 1 h at 37 °C, washed and permeabilized. Nuclei were counterstained with DAPI.

FIG. 7. Amino acid sequence of anti-Nectin2 scFv HRB725 showing CDRs of V_{H} and V_{L} chains; the sequence of the V_{H} chain is underlined, the (G4S)3 linker between the V_{H} and V_{L} chains is in italic, and the V_{L} chain is shown in normal characters.

FIG. 8. Amino acid sequence of anti-PSMA scFv HRB730 showing CDRs of V_{H} and V_{L} chains; the sequence of the V_{H} chain is underlined, the (G4S)3 linker between the V_{H} and V_{L} chains is in italic, and the V_{L} chain is shown in normal characters.

FIG. 9. Anti-Nectin2 scFv-Fc (725v5) is binds to a cancer-exposed epitope of Nectin2. The anti-Nectin2 scFv-Fc (725v5) selectively binds to Nectin 2 when expressed by tumor cells, by contrast with a commercial anti-Nectin2 monoclonal antibody that binds indifferently to normal and tumoral cells expressing Nectin2.

### EXAMPLES

### Example 1: Nectin2 mRNA is Highly Expressed in Prostate Cancer Cells

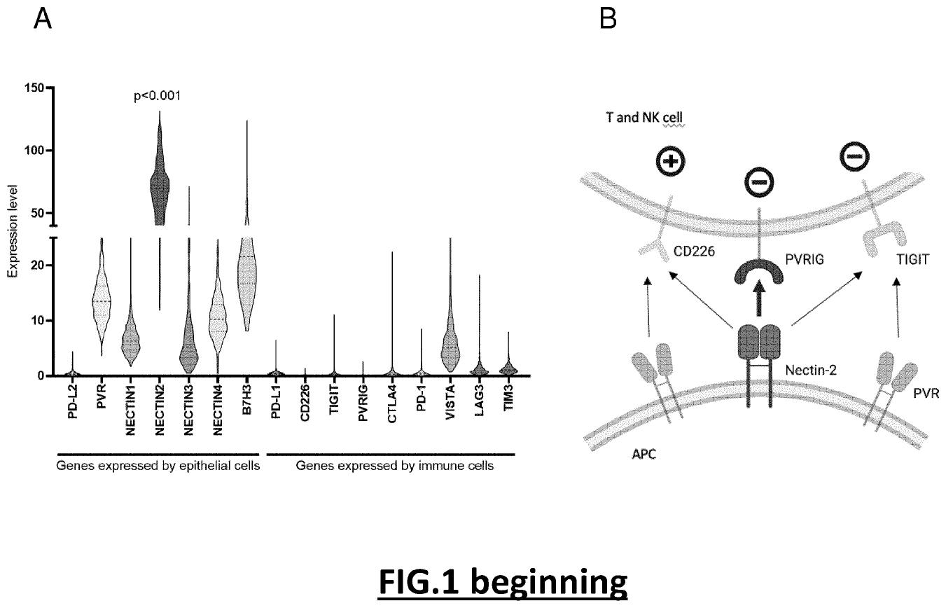

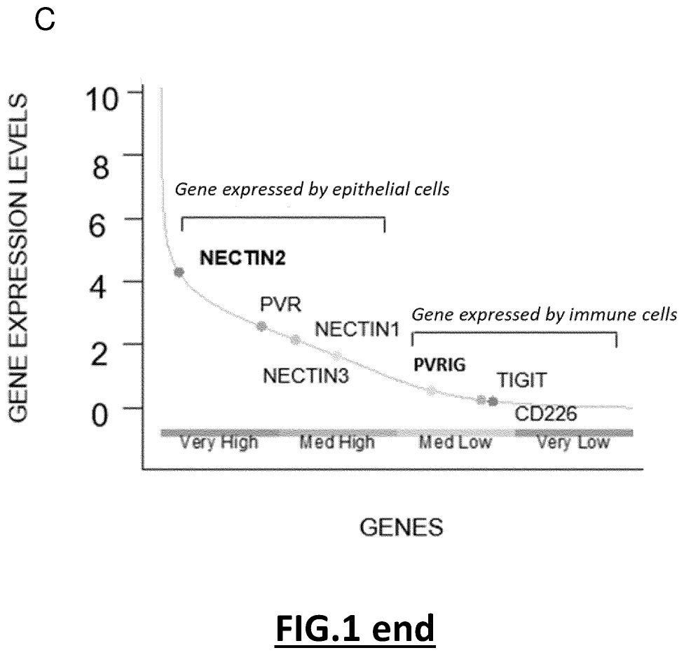

To identify novel targets in prostate cancer we analysed the expression of the most renowned ICs in the publicly available datasets. We compared the mRNA expression levels of genes expressed by epithelial cells and by immune cells using the raw data of the TGCA database from the Human Protein Atlas (https://www.proteinatlas.org/(2022)). Interestingly, as shown in FIG. 1A, we observed a significantly higher expression of Nectin2 in PCa compared to all the other targets, including PD1 and CTLA4.

Nectin2 is a Ca2+-independent cell-cell adhesion molecule, widely expressed on antigen-presenting and tumour cells, whose expression is regulated in tumorigenesis. The role of Nectin2 as an immune checkpoint has already been investigated in breast and ovarian cancer (Oshima, T. et al. Mol. Cancer 12, 60 (2013)). Over-expression of Nectin2 mRNA in prostate cancer tissues was also reported (Oshima, T. et al. Mol. Cancer 12, 60 (2013)). An increase in protein level has been observed in different types of cancer, such as acute myeloid leukaemia, multiple myeloma, gallbladder, and prostate cancer (Sanchez-Correa, B. et al. Immunol. Cell Biol. 90, 109-115 (2012); Casado, J. G. et al. Cancer Immunol. Immunother. CII 58, 1517-1526 (2009); WO2020144697A1). Although primarily a regulator of cell adhesion, motility and proliferation, Nectin2 is also immunomodulatory (Takai, Y., et al. Nat. Rev. Mol. CellBiol. 9, 603-615 (2008)). In fact, this membrane protein can either co-stimulate T and NK cells by interacting with CD226 or suppress their response by binding to the inhibiting receptors TIGIT and PVRIG (Zhu, Y. et al. J. Exp. Med. 213, 167-176 (2016)).

It must be emphasized that another Nectin-family member, PVR, is a ligand of the costimulatory receptor CD226 and the co-inhibitory receptor TIGIT (FIG. 1B). On the other hand, the PVR receptor still seems to be the prevailing ligand for TIGIT in this ligand-receptor network because of the feebleness of the Nectin2-TIGIT interaction (Yu, X. et al. Nat. Immunol. 10, 48-57 (2009)).

However, the Nectin2/TIGIT/PVRIG axis in prostate cancer has not yet been investigated.

### Example 2: PVRIG is the Main Nectin2 Interactor in the Prostate Tumor Microenvironment

To better understand the role of Nectin2 in prostate cancer, we then assessed the expression of its ligand/interactors in bulk RNA-seq data from human prostate cancer samples.

We observed a high expression level of Nectin2 and low expression of TIGIT, thus suggesting that PVRIG is the main Nectin2 interactor in the prostate tumor microenvironment (FIG. 1B). Interestingly, by analyzing the single-cell RNA-seq data from PCa biopsies, we verified that Nectin2 is primarily expressed by epithelial cells and endothelial cells (FIG. 1C). These data were obtained starting from 13 tissue samples from 12 primary tumors and 1 lymph node metastasis, and after standard data processing and quality control procedures they got transcriptomic profiles for 36,424 cells (Chen, S. et al. Nat. Cell Biol. 23, 87-98 (2021)). However, this analysis on primary prostate cancer samples is enriched in epithelial cells. Thus, we checked on lethal PC single cell analysis to validate the PVRIG expression by single cell data. The cohort comprised 2,170 cells from 14 patients and 15 fresh biopsies from three common mCRPC metastatic sites (bone, lymph node and liver). PVRIG was confirmed to be expressed by CD4+ T cells, CD8+ T cells and NK cells (Jordan, M. A. & Wilson, L. Nat. Rev. Cancer 4, 253-265 (2004)). In addition, we confirmed the Nectin2 protein expression in the human prostate cancer sample by immunofluorescence (not shown).

### Example 3: Uprequlation of Nectin2 is Correlated with Senescence

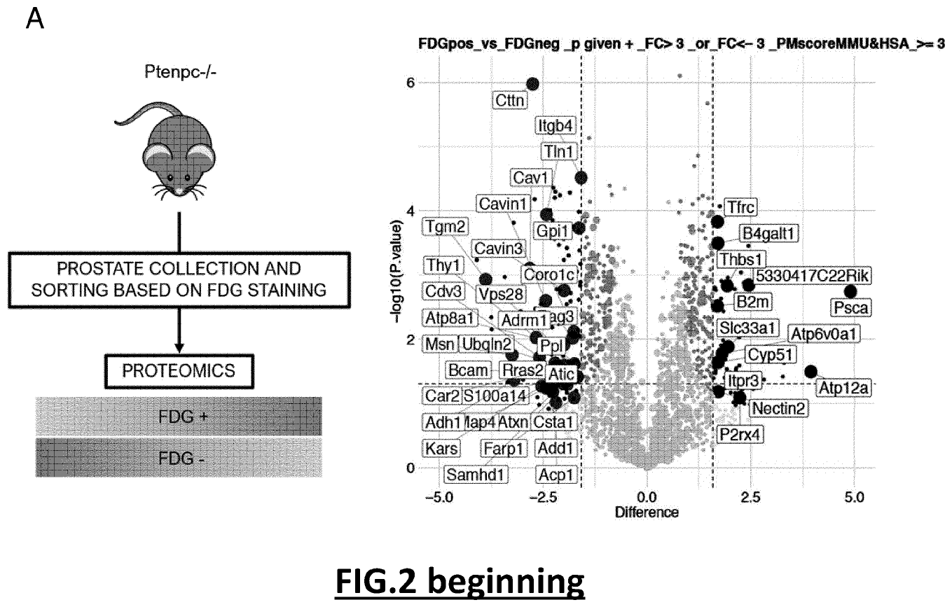

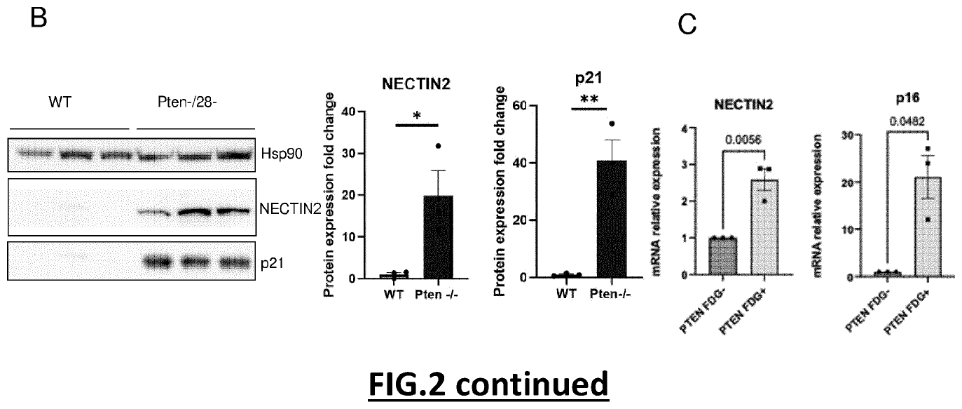

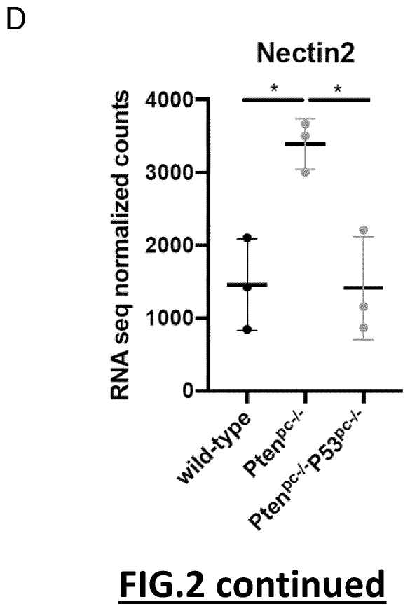

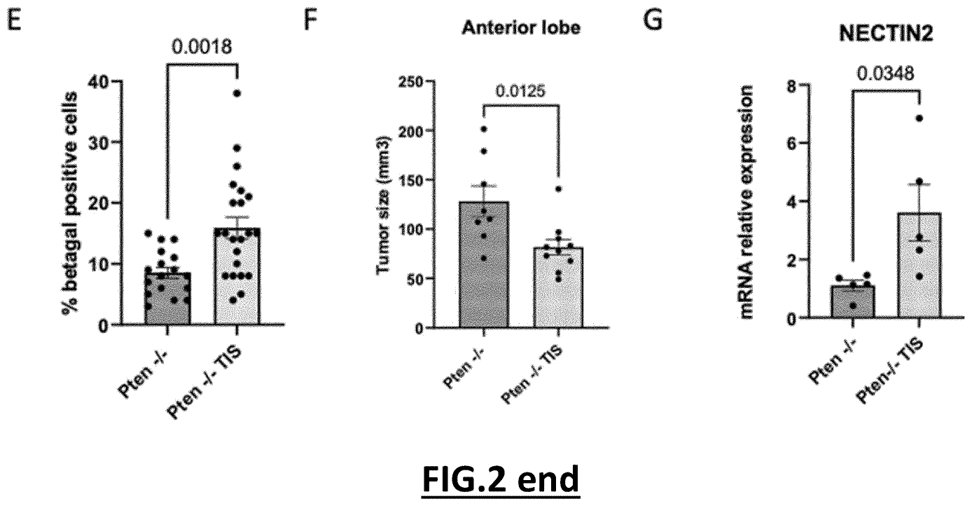

Next, in collaboration with the Proteomic Core Facility of Lausanne at Ecole Polytechnique Fédérale de Lausanne (EPFL), we ran a mass spectrometry and bioinformatic analysis to identify membrane proteins upregulated by the senescent population in Pten^{-/-} mice. Using this technology, we verified that Nectin2 was among the most upregulated transmembrane proteins expressed by senescent cells (FIG. 2A, right panel). Moreover, the bioinformatic analysis revealed that cells having the highest senescence score were also the ones with the highest Nectin2 abundance.

Using our mouse model of Pten-loss-induced cellular senescence (PICS, Toso et al., Cell Reports 9, 75-89 (2014)), we then demonstrated the correlation at both transcriptional and translation levels between senescence and Nectin2 expression. Western blot confirmed this correlation at translation levels in total tumor (FIG. 2B) and this data was also validated by qRT-PCR on sorted C12-FDG+ and C12-FDG− PTEN null tumor cells (FIG. 2C). Besides Nectin2, we also checked for some senescent markers such as p21 (at translational level) and p16 (at transcriptional level), which were upregulated in the non-proliferative cellular fraction. Moreover, results from RNA-seq confirms the Nectin2 upregulation in pten^{-/-} mice when compared to pten^{-/-};p53^{-/-} and wild-type mice (FIG. 2D).

We then verified in vivo whether the upregulation of Nectin2 correlated or not with therapy-induced senescence (TIS) in our genetically engineered mouse model. Mice were treated with Docetaxel 10 mg/Kg once a week for four weeks and, at the end of the treatment, we performed beta-galactosidase staining to confirm senescence induction (FIG. 2E), in line with a reduction of anterior lobe tumour size (FIG. 2F). Real-time PCR data pointed out a significant Nectin2 upregulation (FIG. 2G).

We validated the expression of our target on human prostate cancer cell lines. To test tumor heterogeneity in terms of mutational burden and genetic alteration, we used human prostate cancer cell lines with both a wild-type and mutated form of PTEN and TP53 gene, and in terms of response to androgen receptor signalling.

To assess whether Nectin2 expression was associated with disease progression and resistance to therapy, we treated human PCa cell lines with two common chemotherapy agents used for PCa management to induce cellular senescence (specifically Docetaxel and Palbociclib.) Docetaxel-based regimens have been shown to improve the patients' symptoms and overall survival in mCRPC patient (Mohler, J. L. et al. J. Natl. Compr. Cancer Netw. JNCCN 17, 479-505 (2019)). Besides, Palbociclib (a CDK4/6 inhibitor) is commonly used for treating metastatic prostate cancer in combination with ADT, and it has already been proven to induce senescence in subtoxic concentrations (ClinicalTrials.gov Identifier: NCT02905318).

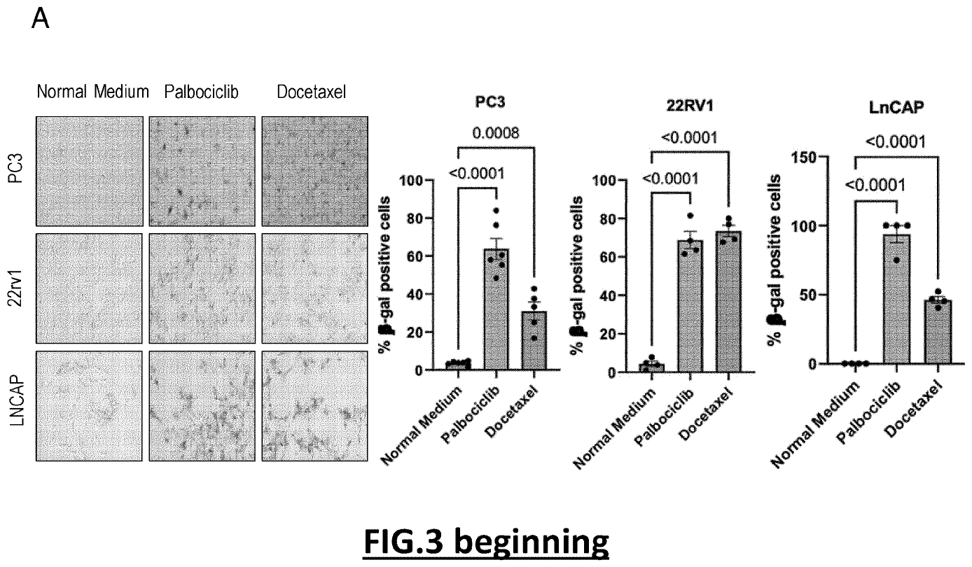

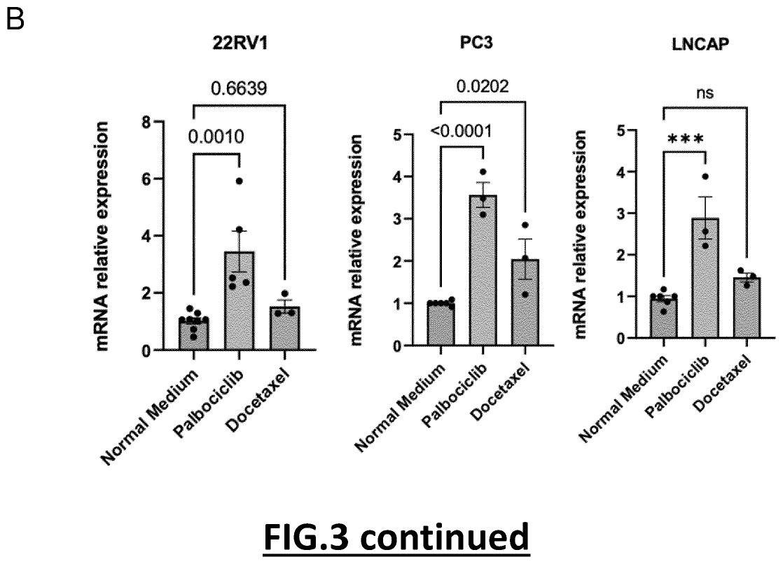

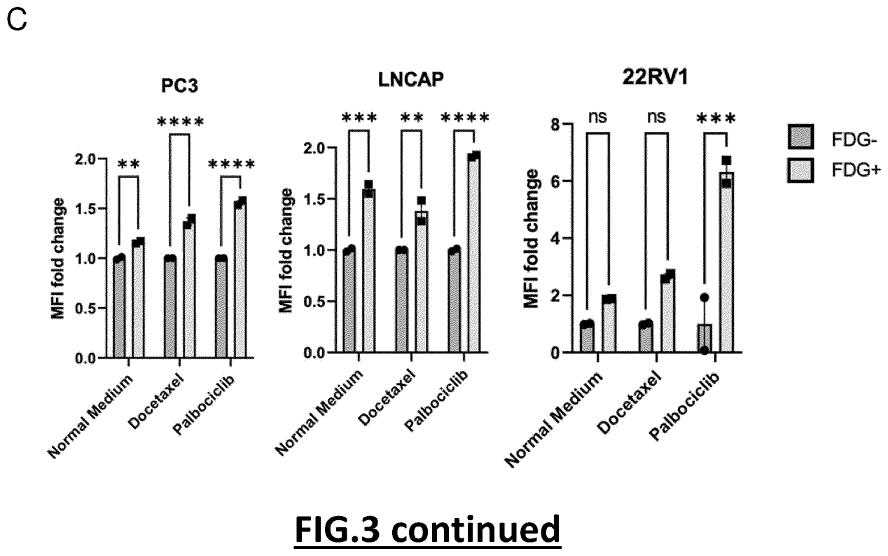

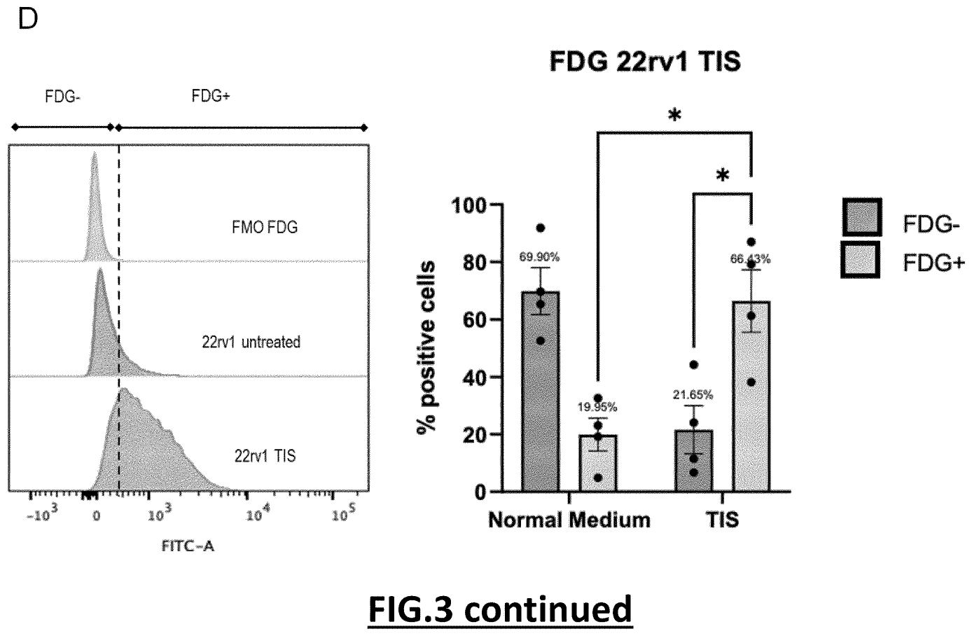

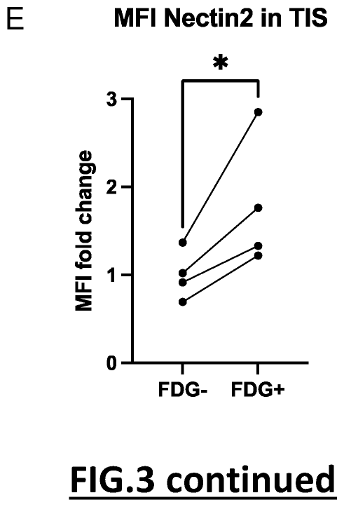

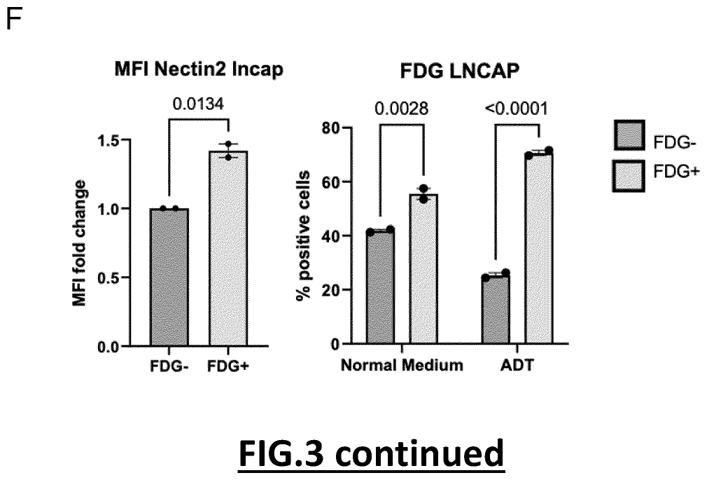

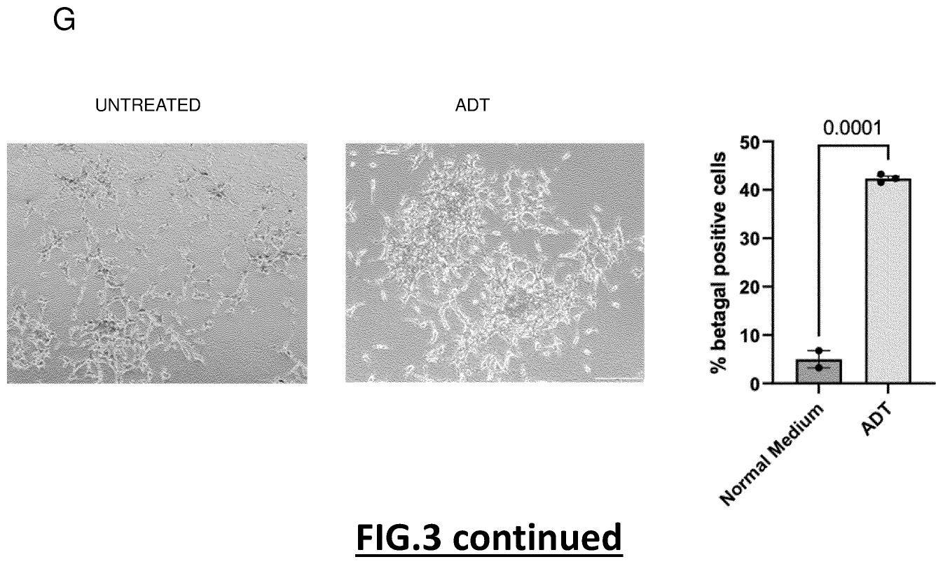

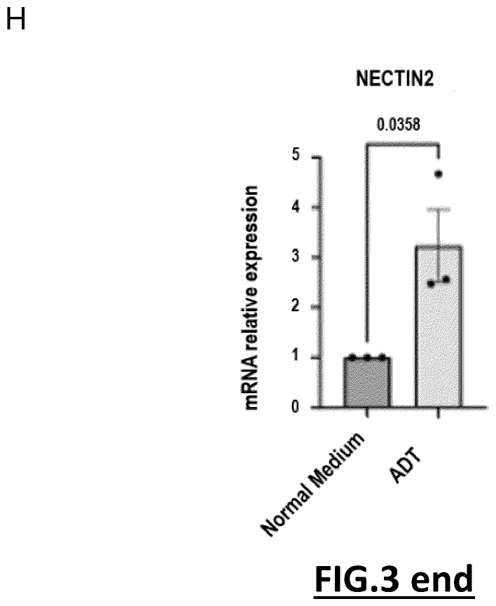

It was also observed that the same senescence induction at subtoxic range is true for Docetaxel and Palbociclib, and the onset of senescent causes resistance to therapy. Later, the efficacy of senescence induction was evaluated using beta-galactosidase assay (FIG. 3A), and real-time PCR was performed on treated and untreated cells to measure Nectin2 mRNA level (FIG. 3B). Treatment with Palbociclib induced a significant increase in the Nectin2 mRNA level in all the tested cell lines. Induction of Nectin2 mRNA levels after Docetaxel treatment was also substantial compared to the untreated population in PC3 and not significant in 22rv1 and LNCAP cells.

Flow cytometry analysis also confirmed the same results, clearly showing upregulation of Nectin2 at the translational level (FIG. 3C) under all the tested conditions.

Senescence induction upon treatment was also confirmed using FDG staining in 22rv1 (FIG. 3D), as well as Nectin2 upregulation in the treated fraction (FIG. 3E).

The induction of senescence was also achieved through treatment with Enzalutamide, to mimic androgen deprivation therapy. For this purpose, we used LNCaP cells, as they express the androgen receptor and, therefore, they are sensitive to the treatment. As demonstrated through FDG staining (not shown) and beta-galactosidase assay (FIG. 3G), the treatment induced senescence and upregulated Nectin2 at both the protein (FIG. 3F) and mRNA levels (FIG. 3H).

### Example 4: Expression of PSMA Positively Correlates with Nectin2 Expression in Senescent Prostate Cancer Cells

Although the expression of Nectin2 appears cell-specific in the prostate TME, we cannot exclude its expression in other organs.

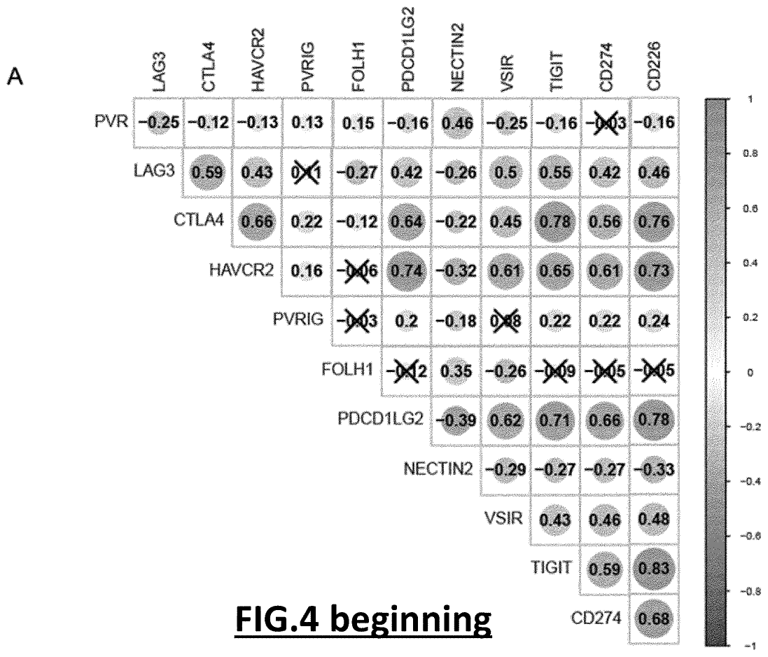

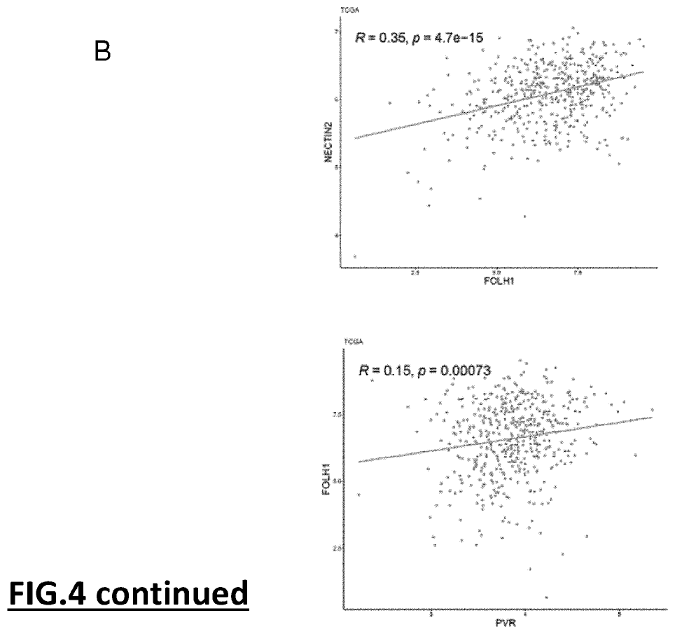

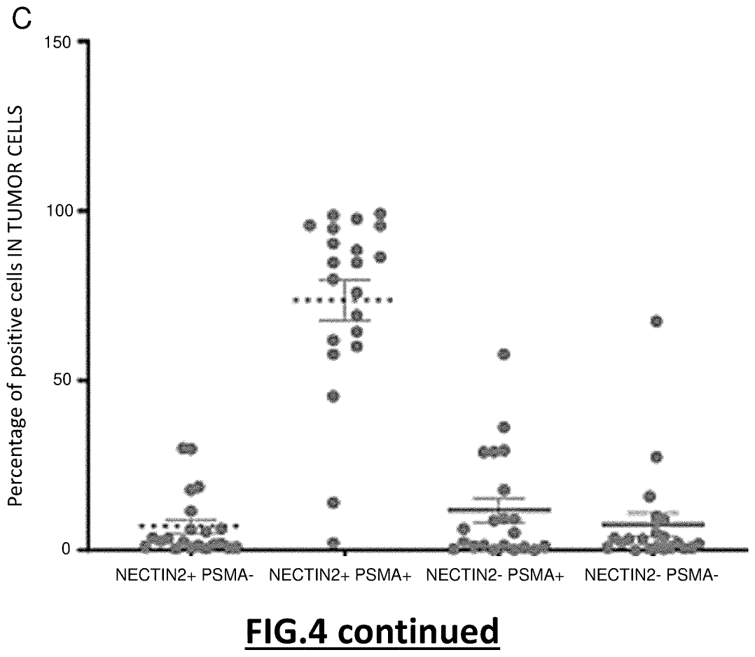

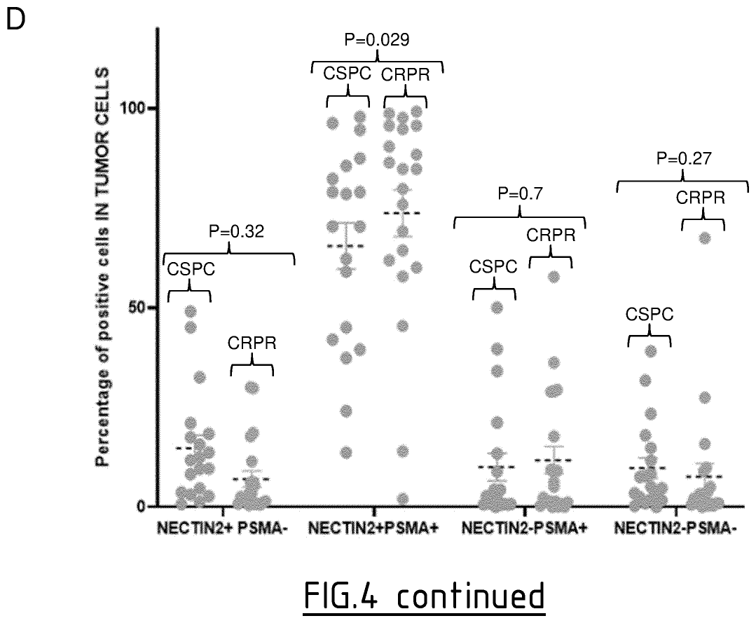

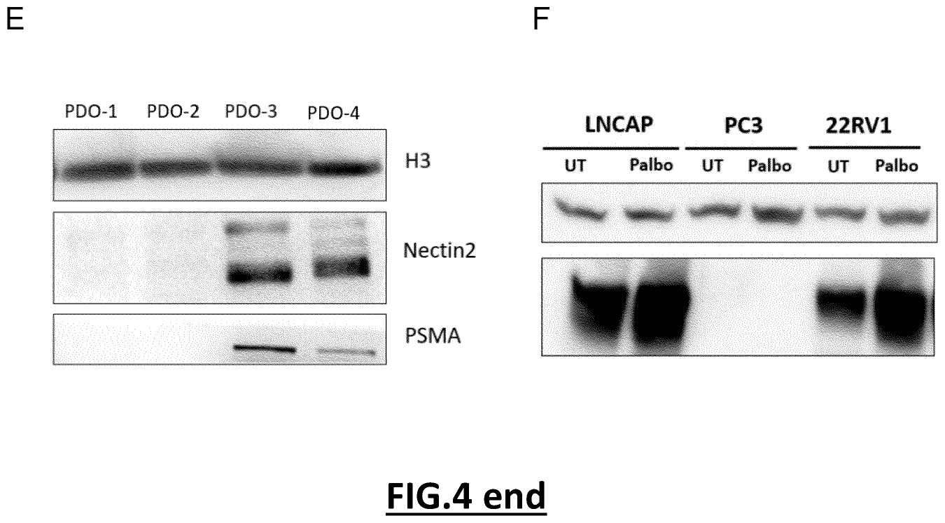

Therefore, we designed a Nectin2− based specific and selective therapeutic strategy by exploiting the prostate-specific membrane antigen (PSMA). To design an effective drug delivery system and immunotherapy, we first checked the correlation of Nectin2 and all the other known immune checkpoints with PSMA. We used the TCGA database and applied a correlation matrix to do that. As shown in FIG. 4A and FIG. 4B, we found that expression of FOLH1 (PSMA) positively correlates only with Nectin2 expression in prostate cancer, which is two times stronger than PVR. Besides, human prostate samples were also analysed by immunofluorescence. In this case, prostate tumour biopsies from normal and tumour tissues were stained using anti-PSMA and anti-Nectin2 primary antibodies. Results of immunofluorescent analysis reveal a higher percentage of epithelial prostate tumour cells co-expressing Nectin2 and PSMA (FIG. 4C). Moreover, samples of castration-sensitive prostate cancer (CSPC) were compared with models of castration-resistant prostate cancer (CRPC) patients, and the percentage of double positive cells was significantly higher in the CRPC ones (FIG. 4D).

Moreover, we performed Western blot analysis on Patient-derived human organoids (PDO), and samples characterized by Nectin2 expression were also positive for PSMA (FIG. 4E).

Despite its clinical exploitation, the regulation of PSMA expression in senescent cells has never been investigated. Thus, we checked PSMA protein levels in our senescence models to evaluate the possibility of targeting it in combination with Nectin2. Western blot of human cell lines treated with TIS showed increased PSMA expression in both LNCaP and 22RV1 cells, while no bands were detected in PC3 and since they do not express PSMA (FIG. 4F).

### Example 5: Senolytic Dual CAR-T Cells Targeting Nectin2 and the Prostate-Specific Membrane Antigen (PSMA)

Nowadays, target therapies in prostate cancer are mainly against the prostate-specific membrane antigen (PSMA). The FOLH1 gene encodes PSMA, a type II transmembrane glycoprotein with folate hydrolase and N-acetylated-alpha-linked-acidic dipeptidase activity. PSMA is expressed in normal, benign and malignant prostate tissues, including intraepithelial and metastatic neoplasia (Hupe, M. C. et al. Front. Oncol. 8, 623 (2018); Troyer, J. et al. Int. J. Cancer 62, 552-558 (1995)). However, it has already been shown that PSMA expression is 100- to 1000-fold times higher in prostatic adenocarcinoma compared to benign prostate (Heston, W. D. W. Urology 49, 104-112 (1997)). For this reason, many radiopharmaceutical and diagnostic tools have been designed to target PSMA.

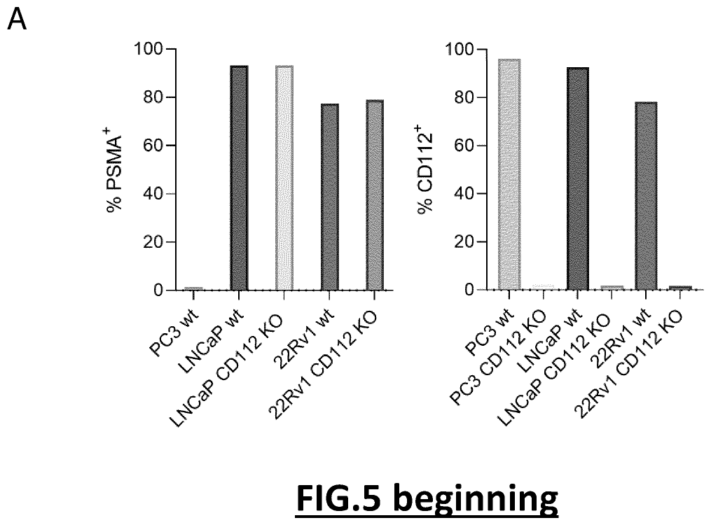

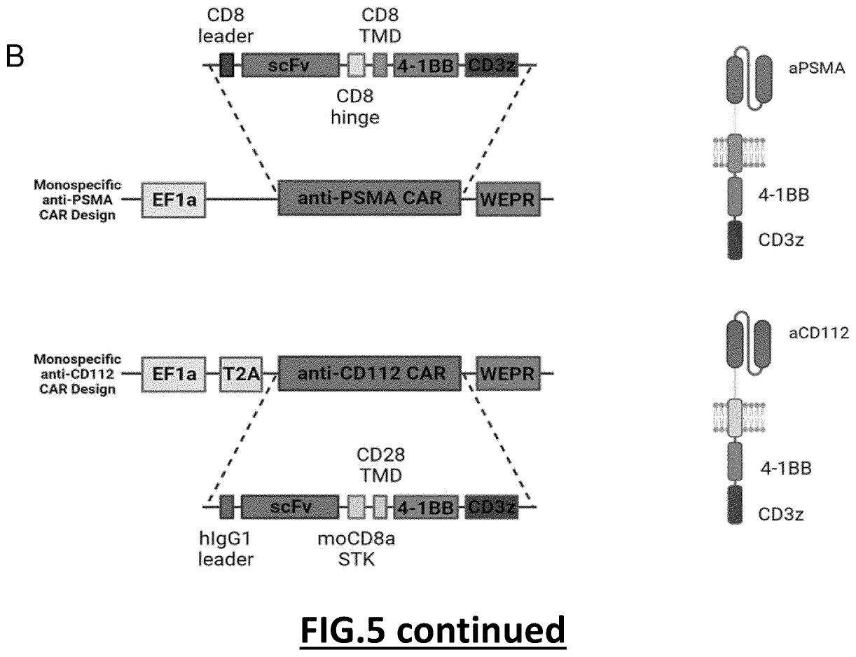

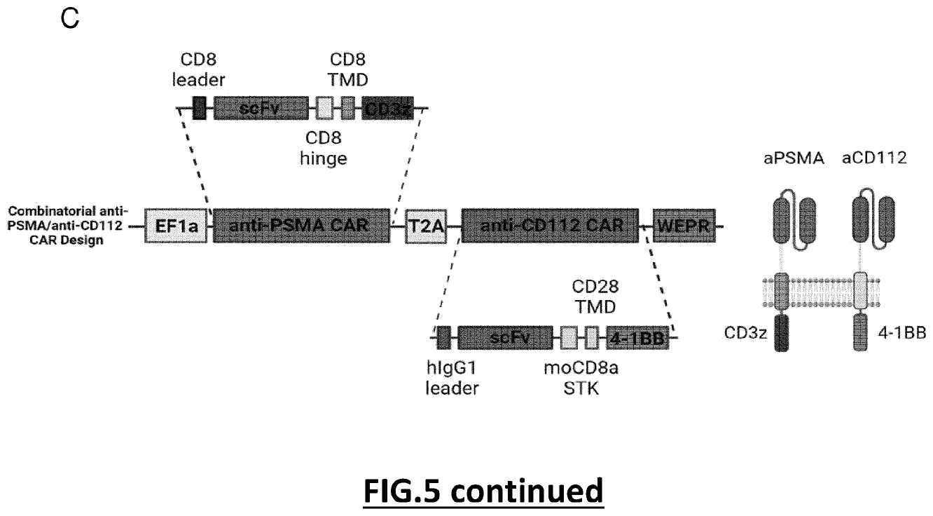

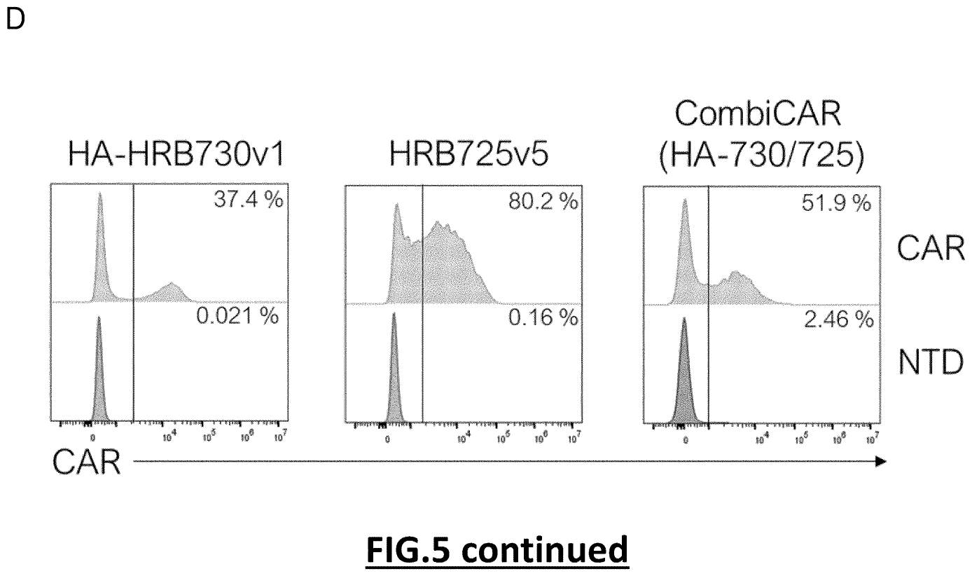

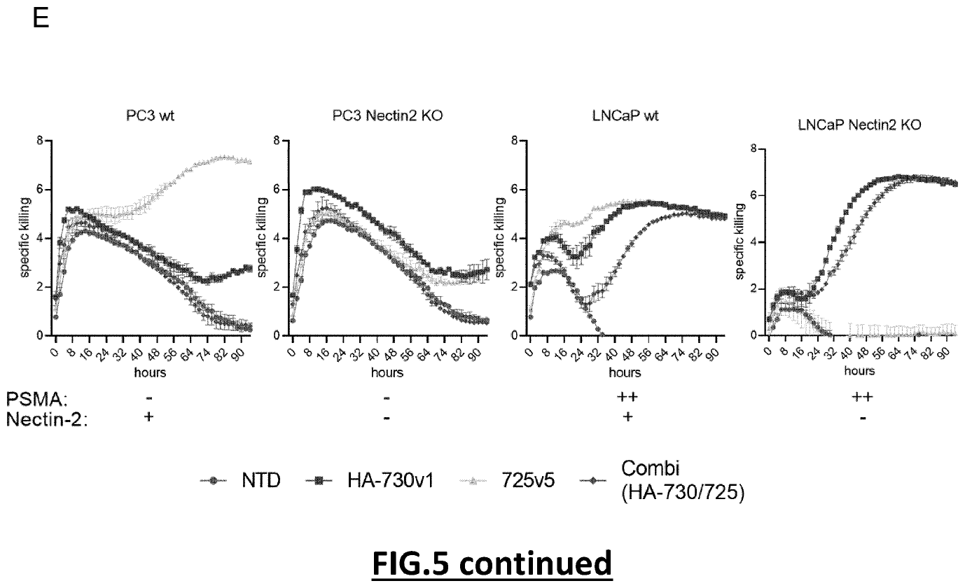

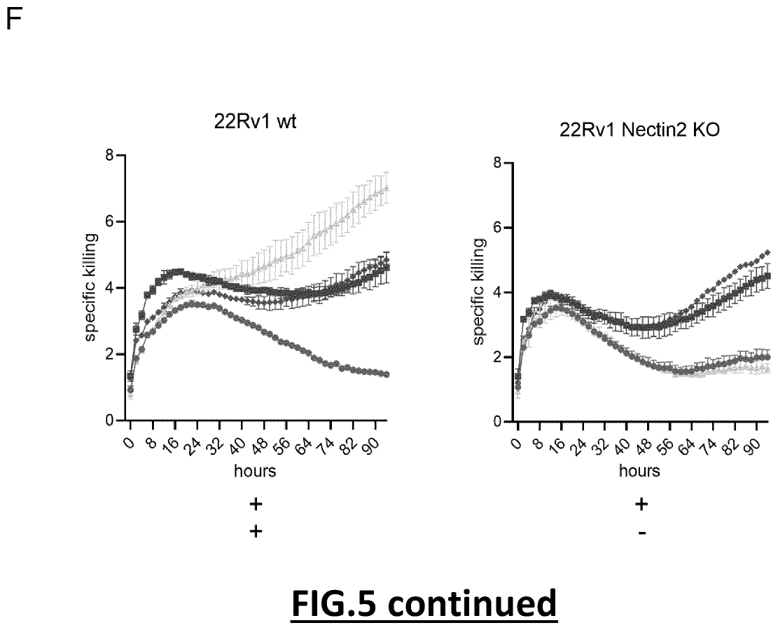

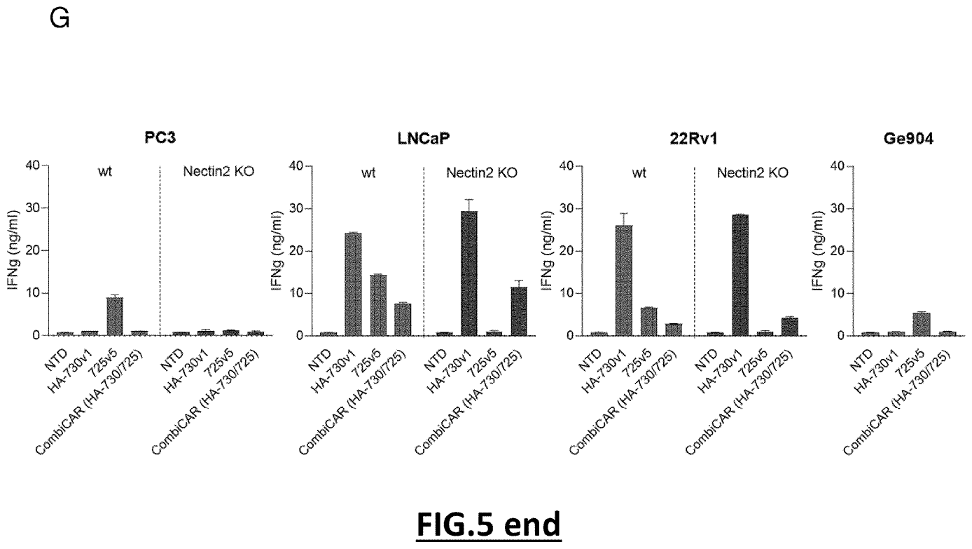

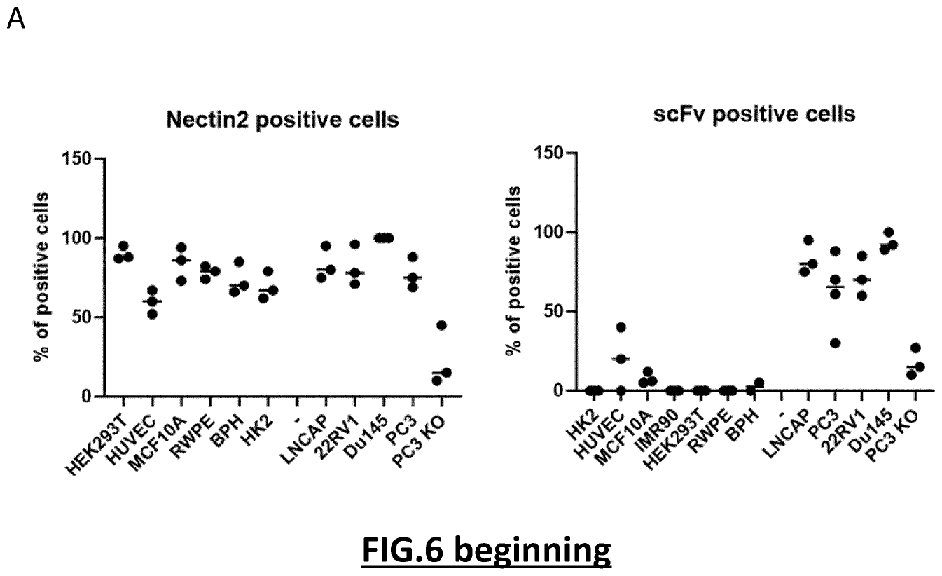

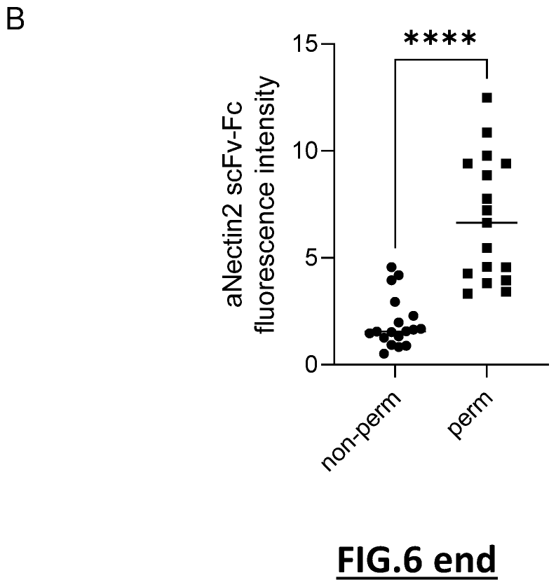

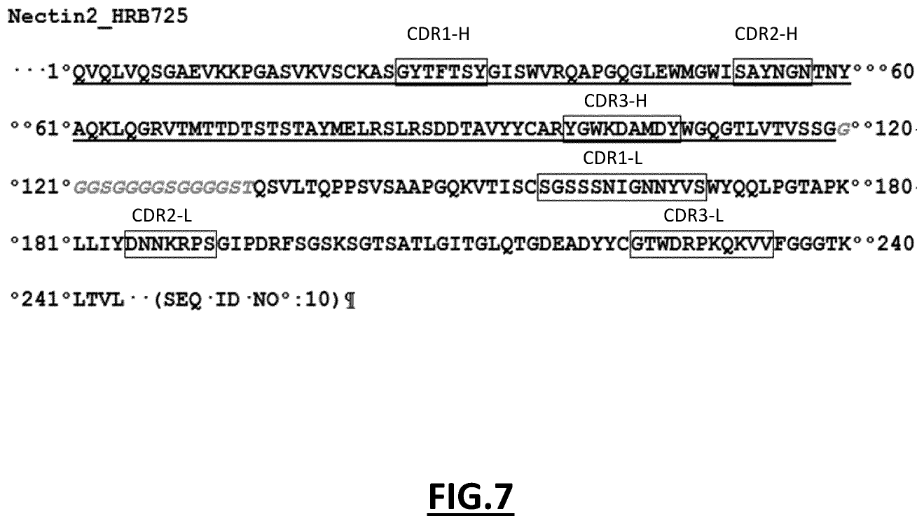

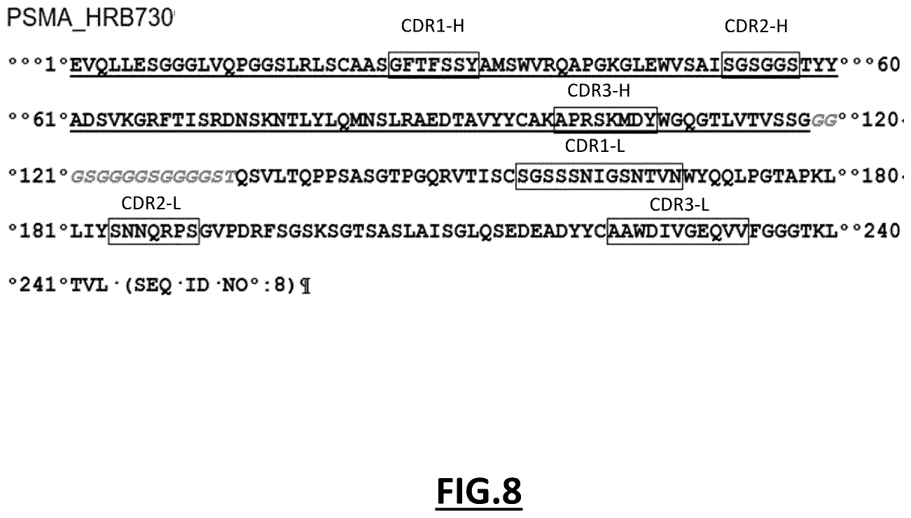

To prevent on-target/off-tumor toxicity with targeting PSMA and Nectin2, we designed a combinatorial CAR T cell approach, a design first described by Kloss et al. to enforce tumor specificity in the absence of tumor-restricted antigens (Kloss, C. et al. Nat. Biotechnol. 31, 71-75 (2013)). Thus, in order to design highly specific and efficient CAR T cells, we screened a phage-display library against PSMA and Nectin2 and generated scFv-Fc constructs with novel anti-PSMA and anti-Nectin2 scFv candidates. To assess their binding specificity to PSMA or Nectin2, we used the PC3 cell line naturally lacking PSMA expression, and generated Nectin2-deficient human PCa cell lines (PC3, LNCaP, 22Rv1) using CRISPR/Cas9 technology. The absence or presence of surface PSMA, as well as the knock-out (KO) of Nectin2 was confirmed via flow cytometry (FIG. 5A). Anti-PSMA (HRB730) (SEQ ID NO: 7-8, FIG. 8) or anti-Nectin2 (HRB725) scFv (SEQ ID NO: 9-10, FIG. 7)—Fc constructs were identified to specifically bind to their target antigens in a dose-dependent manner as assessed via flow cytometry staining of PCa cell lines with anti-PSMA or anti-Nectin2 scFv-Fc constructs in different dilutions (1:1, 1:10, 1:500) and an anti-human Fc-AF488 secondary antibody.

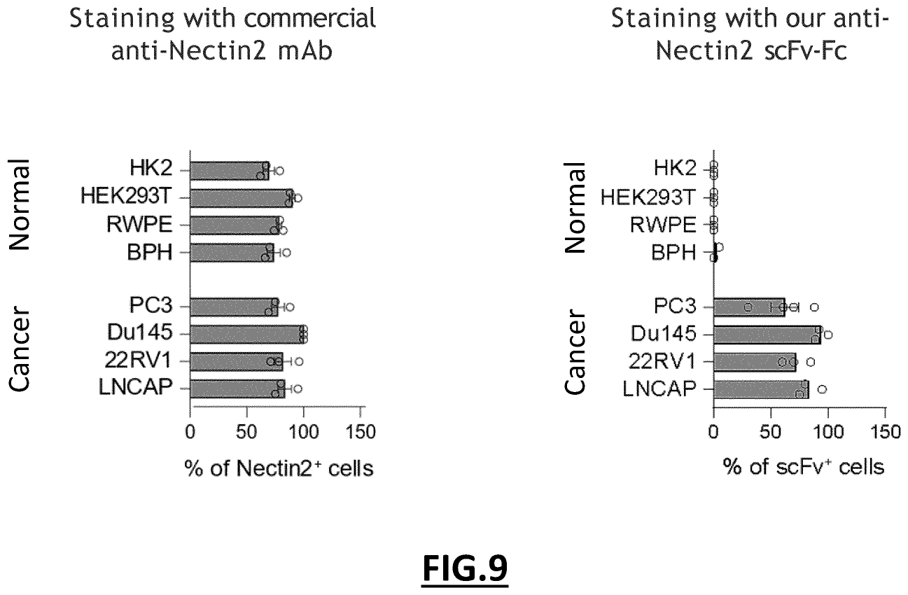

Importantly, the anti-Nectin2 (725v5) scFv-Fc construct was found to be selective of Nectin 2 when expressed on tumor cells, only (FIG. 9). The Nectin2 scFv binds to a cancer-exposed epitope of Nectin2 (comprising or consisting of sequence SEQ ID NO: 28, i.e. amino acids at positions 222-241 of Nectin2 polypeptide as available from Uniprot entry Q92692-1), sparing non-malignant Nectin2-expressing human cells.

Subsequently, we designed and generated monospecific anti-PSMA (SEQ ID NO: 1-2) or anti-Nectin2 (SEQ ID NO: 3-4) CAR constructs (FIG. 5B), as well as a novel CombiCAR construct (SEQ ID NO: 5-6) (FIG. 5C). Here, the PSMA-targeting scFv is linked to a transmembrane domain (TMD), and an intracellular CD3z signaling domain, followed by a self-cleaving T2A peptide, and the anti-Nectin2-targeting scFv linked to a distinct TMD and an intracellular 4-1 BB co-stimulatory domain. Successful T cell activation requires both the intracellular CD3z signaling, as well as the co-stimulatory signal. Thus, this design ensures that both receptors must simultaneously engage to target Nectin2+ cells in order to trigger downstream signaling leading to T cell activation. After T cell transduction with the respective constructs and CAR T cell expansion, CAR expression was assessed via flow cytometry (FIG. 5D). To determine their anti-tumor killing capacities, CAR T cells were co-cultured with human PCa cell lines for 92h (FIG. 5E-F). As shown in FIG. 5E-F, PCa cell lines expressing PSMA are readily lysed by monospecific anti-PSMA CAR T cells, while the PSMA-deficient PCa cell lines PC3 wt and its Nectin2 KO counterpart, as well as the human glioblastoma cell line Ge904, are spared. Similarly, monospecific anti-Nectin2 CAR T cells specifically and efficiently target Nectin2-expressing PCa cell lines. Interestingly, PCa cell lines co-expressing PSMA and Nectin2 are efficiently lysed by CombiCAR T cells, and PSMA-deficient PC3 wt are spared. PSMA₊/Nectin2− PCa cell lines are lysed by CombiCAR T cells as well, as the PSMA-targeting CAR is a first-generation CAR that can be stimulated in vitro by intracellular CD3z signaling domain alone. In vivo, first generation CAR T cells are insufficient to induce an efficient T cell response and persistence and activity is limited (Brocker, T. & Karjalainen, K. J. Exp. Med. 181, 1653-1659 (1995)). In vivo, we expect to target primarily PSMA⁺/Nectin2⁺, and to a lesser extent PSMA⁺/Nectin2⁻, and spare PSMA−/Nectin2^{+/-} PCa cells. Further, as an indicator for successful T cell activation and function, IFNg secretion was assessed in the supernatant post co-culture using sandwich ELISA. As shown in FIG. 5F, the IFNg secretion levels of monospecific anti-PSMA, monospecific anti-Nectin2 and CombiCAR T cells correspond with their lysing capacities in presence of their target antigens, as assessed in FIG. 5G.

1-21. (canceled)

22. An anti-Nectin2 antibody, or antigen binding fragment thereof, that comprises (i) a variable heavy chain domain (V_{H}) comprising a CDR1-H of sequence SEQ ID NO: 11, a CDR2-H of sequence SEQ ID NO: 12, and a CDR3-H of sequence SEQ ID NO: 13, and (ii) a variable light chain domain (V_{L}) comprising a CDR1-L of sequence SEQ ID NO: 14, a CDR2-L of sequence SEQ ID NO: 15, and a CDR3-L of sequence SEQ ID NO: 16.

23. The anti-Nectin2 antibody, or antigen binding fragment thereof, according to claim 22, which V_{H} comprises sequence SEQ ID NO: 17 or a sequence at least 80% identical thereto, and/or which V_{L} comprises sequence SEQ ID NO: 18 or a sequence at least 80% identical thereto.

24. The anti-Nectin2 antibody, or antigen binding fragment thereof, according to claim 22, which is a single chain variable fragment (scFv).

25. The anti-Nectin2 antibody, or antigen binding fragment thereof, according to claim 24, which comprises or consists of sequence SEQ ID NO: 10.

26. A polypeptide comprising the anti-Nectin2 scFv according to claim 24.

27. A pair of isolated nucleic acids comprising a sequence encoding respectively the variable heavy chain domain (V_{H}) and variable light chain domain (V_{L}) of the anti-Nectin2 antibody, or antigen binding fragment thereof according to claim 22.

28. An isolated nucleic acid comprising a sequence encoding the anti-Nectin2 scFv according to claim 24 or a polypeptide comprising an anti-Nectin2 scFv, wherein the anti-Nectin2 scFv is a single chain variable fragment.

29. An antibody-drug conjugate (ADC) comprising (i) an anti-Nectin2 antibody, or antigen binding fragment thereof, according to claim 22, (ii) a cytotoxic payload, and (iii) a linker connecting the anti-Nectin2 antibody, or antigen binding fragment thereof, and the cytotoxic payload.

30. A method of treating cancer comprising administering an antibody-drug conjugate (ADC) as defined in claim 29 in a subject in need thereof.

31. The method according to claim 30, wherein the cancer is prostate cancer.

32. An anti-PSMA antibody, or antigen binding fragment thereof, that comprises (i) a variable heavy chain domain (V_{H}) comprising a CDR1-H of sequence SEQ ID NO: 20, a CDR2-H of sequence SEQ ID NO: 21, and a CDR3-H of sequence SEQ ID NO: 22, and (ii) a variable light chain domain (V_{L}) comprising a CDR1-L of sequence SEQ ID NO: 23, a CDR2-L of sequence SEQ ID NO: 24, and a CDR3-L of sequence SEQ ID NO: 25.

33. The anti-PSMA antibody, or antigen binding fragment thereof, according to claim 32, which V_{H} comprises sequence SEQ ID NO: 26 or a sequence at least 80% identical thereto, and/or which V_{L} comprises sequence SEQ ID NO: 27 or a sequence at least 80% identical thereto.

34. The anti-PSMA antibody, or antigen binding fragment thereof, according to claim 33, which is a single chain variable fragment (scFv).

35. The anti-PSMA antibody, or antigen binding fragment thereof, according to claim 34, which comprises or consists of sequence SEQ ID NO: 8.

36. A pair of isolated nucleic acids comprising a sequence encoding respectively the variable heavy chain domain (V_{H}) and variable light chain domain (V_{L}) of the anti-PSMA antibody, or antigen binding fragment thereof, according to claim 32.

37. An isolated nucleic acid comprising a sequence encoding the anti-PSMA scFv according to claim 34.

38. An antibody-drug conjugate (ADC) comprising (i) an anti-PSMA antibody, or antigen binding fragment thereof, according to claim 32, (ii) a cytotoxic payload, and (iii) a linker connecting the anti-PSMA antibody, or antigen binding fragment thereof, and the cytotoxic payload.

39. A method of treating cancer comprising administering the antibody-drug conjugate (ADC) as defined in claim 38 in a subject in need thereof.

40. A bispecific antibody construct that binds at least to Nectin2 and PSMA.

41. A bispecific antibody construct according to claim 40 that comprises
an anti-Nectin2 antibody, or antigen binding fragment thereof comprising a variable heavy chain domain (V_{H}) comprising SEQ ID NO: 17 or a sequence at least 80% identical thereto, and/or a variable light chain domain (V_{L}) comprising SEQ ID NO: 18 or a sequence at least 80% identical thereto, wherein the anti-Nectin2 antibody, or antigen binding fragment thereof is a single chain variable fragment (scFv) and
an anti-PSMA antibody, or antigen binding fragment thereof comprising a variable heavy chain domain (V_{H}) comprising SEQ ID NO: 26 or a sequence at least 80% identical thereto, and/or a variable light chain domain (V_{L}) comprising SEQ ID NO: 27 or a sequence at least 80% identical thereto, wherein the anti-Nectin2 antibody, or antigen binding fragment thereof is a single chain variable fragment (scFv).

42. An antibody-drug conjugate (ADC) comprising (i) a bispecific antibody construct that binds to Nectin2 and PSMA as defined in claim 40, (ii) a cytotoxic payload, and (iii) a linker connecting the bispecific antibody construct and the cytotoxic payload.

43. A method of treating cancer comprising administering an antibody-drug conjugate (ADC) as defined in claim 42 in a subject in need thereof.

44. A pharmaceutical composition comprising an antibody-drug conjugate (ADC), and a pharmaceutically acceptable carrier, wherein the ADC comprises:
A. (a) an anti-Nectin2 antibody, or antigen binding fragment thereof, (b) a cytotoxic payload, and (c) a linker connecting the anti-Nectin2 antibody, or antigen binding fragment thereof, and the cytotoxic payload, wherein the anti-Nectin2 antibody, or antigen binding fragment thereof comprises:
(i) a variable heavy chain domain (V_{H}) comprising a CDR1-H of sequence SEQ ID NO: 11, a CDR2-H of sequence SEQ ID NO: 12, and a CDR3-H of sequence SEQ ID NO: 13, and
(ii) a variable light chain domain (V_{L}) comprising a CDR1-L of sequence SEQ ID NO: 14, a CDR2-L of sequence SEQ ID NO: 15, and a CDR3-L of sequence SEQ ID NO: 16;
B. (a) an anti-PSMA antibody, or antigen binding fragment thereof, (b) a cytotoxic payload, and (c) a linker connecting the anti-PSMA antibody, or antigen binding fragment thereof, and the cytotoxic payload, wherein the anti-PSMA antibody, or antigen binding fragment thereof comprises:
(i) a variable heavy chain domain (V_{H}) comprising a CDR1-H of sequence SEQ ID NO: 20, a CDR2-H of sequence SEQ ID NO: 21, and a CDR3-H of sequence SEQ ID NO: 22, and
(ii) a variable light chain domain (V_{L}) comprising a CDR1-L of sequence SEQ ID NO: 23, a CDR2-L of sequence SEQ ID NO: 24, and a CDR3-L of sequence SEQ ID NO: 25; or
C. the bispecific antibody construct of claim 41.
